(19) United States
(12) Patent Application Publication
(10) Pub. No.: US 2003/0138433 A1
(43) Pub. Date: Jul. 24, 2003

(54) Autologous adoptive immunotherapy with antigen-specific primed T cells or B cells to promote antigen-specific immune responses with an appropriate cytokine bias

(76) Inventors: Martha K. Newell, Colorado Springs, CO (US); William F. Wade, Lebanon, NH (US)

Correspondence Address: Konstantinos Andrikopoulos, Esq., Ph.D., Wolf, Greenfield & Sacks, P.C., 600 Atlantic Avenue, Boston, MA, 02210, US

(21) Appl. No.: 10/225,313

(22) Filed: Aug. 21, 2002

Related U.S. Application Data
(63) Continuation of application No. 09/596,387, filed on Jun. 16, 2000, now abandoned.
(60) Provisional application No. 60/139,925, filed on Jun. 17, 1999.

(51) Int. Cl. A61K 45/00; A61K 39/395; A61K 38/20

(52) U.S. Cl. 424/178.1; 424/93.7; 424/85.2

(57) ABSTRACT

The invention relates to methods and compositions for promoting antigen-specific immune responses with an appropriate cytokine bias. More specifically, the present invention relates to methods of using autologous adoptive immunotherapy with antigen-specific, ex vivo-primed T cells or B cells to promote antigen-specific immune responses with a Th1 or Th2 cytokine bias.

### RELATED APPLICATIONS

This application is a continuation of U.S. Ser. No.: 09/596,387, filed Jun. 16, 2000, which claims priority under 35 U.S.C. §119(e) from Provisional U.S. Patent Application Serial No. 60/139,925 filed Jun. 17, 1999, both applications entitled AUTOLOGOUS ADOPTIVE IMMUNOTHERAPY WITH ANTIGEN-SPECIFIC PRIMED T CELLS OR B CELLS TO PROMOTE ANTIGEN-SPECIFIC IMMUNE RESPONSES WITH AN APPROPRIATE CYTOKINE BIAS. The contents of the both applications are hereby expressly incorporated by reference.

### GOVERNMENT SUPPORT

This work was funded in part by grant number AI31160 from the National Institutes of Health. Accordingly, the United States Government may have certain rights to this invention.

### FIELD OF THE INVENTION

This invention relates to methods and compositions for promoting antigen-specific immune responses with an appropriate cytokine bias. More specifically, the present invention relates to methods of using autologous adoptive immunotherapy with antigen-specific, ex vivo-primed T cells or B cells to promote antigen-specific immune responses with a Th1 or Th2 cytokine bias.

### BACKGROUND OF THE INVENTION

A characteristic of the immune system is the specific recognition of antigens. This includes the ability to discriminate between self and non-self antigens and a memory-like potential that enables a fast and specific reaction to previously encountered antigens. The vertebrate immune system reacts to foreign and tumor antigens with a cascade of molecular and cellular events that ultimately results in the humoral and cell-mediated immune response.

The major pathway of the immune defense involving antigen-specific recognition commences with the trapping of the antigen by antigen presenting cells (APCs), and the subsequent migration of these cells to lymphoid organs (e.g., thymus). There, the APCs present antigen to subclasses of T cells classified as mature T helper cells. Upon specific recognition of the presented antigen, the mature T helper cells can be triggered to become activated T helper cells. The activated T helper cells regulate both the humoral immune response by inducing the differentiation of mature B cells to antibody producing plasma cells and the cell-mediated immune response by activation of mature cytotoxic T cells.

Dendritic cells (DCs) are considered to be the most efficient APCs since fewer DCs are required to induce an optimal T cell immune response. In addition to their capacity to present antigen, DCs are also highly efficient at antigen capture, processing, and migration. Therefore, DCs have been selected as the “APC of choice” to generate antigen-specific T cells for immunotherapy. Since DCs constitute only 0.1-0.5% of human peripheral blood (PB) mononuclear cells, considerable difficulty and expense has been experienced in obtaining sufficient numbers of highly enriched mature DCs (Girolomoni and Ricciardicastagnoli, 1997, Immunol Today, 18:102-104).

T lymphocytes recognize antigen in the context of the Major Histocompatibility Complex (MHC) molecules by means of the T cell receptor (TCR) expressed on their cell surface. The TCR is a disulfide linked heterodimer noncovalently associated with the CD3 complex (Allison, J. P., et al., Ann. Rev. Immunol., 1987, 5:503). Most T cells carry TCRs consisting of α and β glycoproteins. T cells use mechanisms to generate diversity in their receptor molecules similar to those operating in B cells (Kronenberg, M., et al., Ann. Rev. Immunol., 1986, 4:529; Tonegawa S., Nature, 1983, 302:575).

Two different types of T cells are involved in antigen recognition within the MHC context. Mature T helper cells (CD4⁺8^{lo}) recognize antigen in the context of class II MHC molecules, whereas cytotoxic T cells (CD4^{lo}8⁺) recognize antigen in the context of class I MHC determinants (Swain, S. L., Immun. Rev., 1983, 74:129-142; Dialynas, P. D., et al., Immun. Rev., 1983, 74:29-56).

One of the ways the immune system regulates itself is through the selective activation of inflammatory (Th1-response) or helper T cells (Th2-response). During a Th2 response, a number of lymphokines are known to be produced by the Th2 cells. Typically, in a Th2 response interleukins IL-4, IL-5, IL-6, IL-9, IL-10, and IL-13 are produced, and are primarily involved in providing optimal help for humoral immune responses such as IgE and IgG4 antibody isotype switching (Mosmann, 1989, Annu. Rev. Immunol., 7:145-173). A Th1 response on the other hand is associated with the production of IL-2, tumor necrosis factor α (TNF-α), interferon-γ (IFN-γ), and lymphotoxin production. Th1 cells are primarily responsible for cell-mediated immunity such as delayed type hypersensitivity, and/or inflammatory responses. Some pathogens elicit polarized CD4⁺ helper T cell responses in vitro, suggesting that polarized subsets can be generated in vivo (Scott et al., 1989, Immunol Rev, 112:181-182). Production of an inappropriate pattern(s) of cytokines in response to a pathogen can lead to aggravation rather than cure of the disease. Th1-mediated inflammatory and cytotoxic responses, for example, seem to be required for the clearing of intracellular pathogens effectively, while development of a Th2-polarized response contributes to increased pathogenesis of Leishmaniasis (Sadick et al., 1990, J Exp Med, 171:115-127), leprocy (Yamamura, 1992, Science, 255:12) and, possibly, AIDS (Clerici and Shearer, 1993, Immunol Today, 14:107-111). Current vaccination protocols, therefore, may not be as effective in mounting responses with a specific cytokine bias for the treatment/prevention of cytokine/antigen-specific disorders.

Primary encounter with antigens stimulates specific B cells not only to differentiate into cells that produce antibody at a high rate (plasma cells), but also to give rise to populations of memory cells. These cells have many characteristics that differ from naive B cells (cells that have not encountered antigen), including their lifespan (Strober, 1975, Transplant Rev, 24, 84-112). When re-exposed to antigen, memory cells generate secondary IgG responses that are enhanced in rate, titre and affinity. Such cells are considered as small resting lymphocytes which survive for long periods in a quiescent state between each antigen encounter. Similarly, the majority of mature peripheral CD4⁺ helper T cells are resting and can also be divided into naive and memory subsets.

There exists a need to provide long term memory lymphocyte cells for antigens that elicit specific Th1 or Th2 responses.

There also exists a need to provide reagents for effective immune responses with an appropriate Th1 or Th2 cytokine bias.

### SUMMARY OF THE INVENTION

The invention, in one important part, provides methods and compositions for promoting antigen-specific immune responses with an appropriate cytokine bias. More specifically, the present invention relates to methods of using autologous adoptive immunotherapy with antigen-specific, ex vivo-primed T cells or B cells to promote antigen-specific immune responses with Th1 or Th2 cytokine bias.

Surprisingly, according to the invention, it has been discovered that the ex vivo treatment of lymphocytes present in a relatively small volume of a blood sample, given an appropriate antigenic stimulation, can cause the lymphocytes to develop properties similar to those present in very efficient APCs, making their reinfusion into the subject a significant source of long-lived memory cells. This is in contrast to a direct, in vivo administration of an antigen which results in the generation of relatively few antigen-specific APCs.

According to one aspect of the invention, a method of autologous adoptive immunotherapy, is provided. The method involves obtaining B cells that have been originally isolated from a subject and subsequently contacted in vitro with a target antigen-conjugate to produce target antigen manipulated B cells, and infusing the target antigen manipulated B cells into the subject. The target antigen conjugate, comprises a target antigen that elicits a Th2 response, conjugated to an antibody, a fragment of an antibody, a peptide, and/or a molecule that selectively binds a B cell cell-surface immunoglobulin. In important embodiments, the target antigen manipulated B cells enhance an antigen-specific immune cell response to the target antigen that elicits a Th2 response in a subject. In some embodiments, sources of antigens that elicit a Th2 response include, but are not limited to, extracellular parasites (e.g., Entamoeba Histolytica, Giardia Lamblia, hookworms and other helminths, etc.), Cornebacterium Diptheriae, Vibrio Cholerae, Neisseria Meningitidis, and Staphylococcus Aureus. In certain embodiments, stimulation of naive T cells of the subject with target antigen manipulated B cells occurs in vitro, prior to the infusion of target antigen manipulated B cells into the subject. The in vitro stimulated naive T cells may then be returned into the subject. In further embodiments, if stimulation of naive T cells of the subject with target antigen manipulated B cells occurs in vitro, the target antigen manipulated B cells may not be returned into the subject with the in vitro stimulated naive T cells. In some embodiments, the B cells can be isolated from peripheral blood, or from an in vitro hematopoietic progenitor cell culture. In certain embodiments, the in vitro contacting of the isolated B cells with a target antigen conjugate, further comprises contacting the isolated B cells with an agent selected from the group consisting of a B cell co-stimulating agent and a Th2 cytokine. In preferred embodiments, the B cell co-stimulating agent can be TSA-1, CD2, CD5, CD24, CD28, CD40L, CD49a, CD80, CD81 and/or CD86, and the Th2 cytokine can be IL-4, IL-5, IL-6, lL-9, IL-10, and/or IL-13.

According to another aspect of the invention, a method of autologous adoptive immunotherapy, is provided. The method involves obtaining CD4⁺ helper T cells that have been originally isolated from a subject and subsequently contacted in vitro with a target antigen-conjugate to produce target antigen manipulated CD4⁺ helper T cells, and infusing the target antigen manipulated CD4⁺ helper T cells into the subject. The target antigen conjugate, comprises a target antigen that elicits a Th2 response, conjugated to an antibody, a fragment of an antibody, a peptide, and/or a molecule that selectively binds a T cell receptor. In important embodiments, the target antigen manipulated CD4⁺ helper T cells enhance an antigen-specific immune cell response to the target antigen that elicits a Th2 response in a subject. In some embodiments, sources of antigens that elicit a Th2 response include, but are not limited to, extracellular parasites (e.g., Entamoeba Histolytica, Giardia Lamblia, hookworms and other helminths, etc.), Cornebacterium Diptheriae, Vibrio Cholerae, Neisseria Meningitidis, and Staphylococcus Aureus. In certain embodiments, stimulation of naive T cells of the subject with target antigen manipulated CD4⁺ helper T cells occurs in vitro, prior to the infusion of target antigen manipulated CD4⁺ helper T cells into the subject. The in vitro stimulated naive T cells may then be returned into the subject. In some embodiments, the CD4⁺ helper T cells are isolated from peripheral blood and/or an in vitro hematopoietic progenitor cell culture. In certain embodiments, the in vitro contacting of isolated CD4⁺ helper T cells with a target antigen conjugate, further comprises contacting the isolated CD4⁺ helper T cells with a Th2 cytokine. In preferred embodiments, the Th2 cytokine can be IL-4, IL-5, IL-6, IL-9, IL-10, and/or IL-13.

According to another aspect of the invention, a method of autologous adoptive immunotherapy, is provided. The method involves obtaining CD4⁺ helper T cells that have been originally isolated from a subject and subsequently contacted in vitro with a target antigen-conjugate to produce target antigen manipulated CD4⁺ helper T cells, and infusing the target antigen manipulated CD4⁺ helper T cells into the subject. The target antigen conjugate, comprises a target antigen that elicits a Th1 response, conjugated to a bi-specific antibody, a fragment of an antibody, a peptide, and/or a molecule that selectively binds both a T cell receptor and CD4 on the same CD4⁺ helper T cells. In important embodiments, the target antigen manipulated CD4⁺ helper T cells enhance an antigen-specific immune cell response to the target antigen that elicits a Th1 response in a subject. In some embodiments, sources of antigens that elicit a Th1 response include, but are not limited to, mycobacteria (e.g., tubercolosis, leprae), intracellular pathogens (protozoans: Leishmania, Toxoplasma, Trypanosoma, Plasmodium), Schistosoma, Trichiella Spiralis, Salmonella Typhimurium, HIV, Hepatitis C, Haemophilus Influenza, antigens presented by hepatic non-parenchymal cells (e.g., Kupfer cells), OVA-conjugates, antigens that cause contact hypersensitivity (e.g., nickel, arylates, poison ivy, poison oak), allergans (e.g., birch pollen). In certain embodiments, stimulation of naive T cells of the subject with target antigen manipulated CD4⁺ helper T cells occurs in vitro, prior to the infusion of target antigen manipulated CD4⁺ helper T cells into the subject. In some embodiments, the CD4⁺ helper T cells are isolated from peripheral blood and/or an in vitro hematopoietic progenitor cell culture. In certain embodiments, the in vitro contacting of the isolated CD4⁺ helper T cells with a target antigen conjugate, further comprises contacting the isolated CD4⁺ helper T cells with a Th1 cytokine. In preferred embodiments, the Th1 cytokine can be IL-2, TNF-α, and IFN-γ.

The invention also provides target antigen-specific immune cell response enhancing compositions. According to one aspect, the composition comprises a target antigen conjugated to an antibody that selectively binds a B cell cell-surface immunoglobulin, wherein the target antigen elicits a Th2 response. According to another aspect, the composition comprises a target antigen conjugated to an antibody that selectively binds a T cell receptor, wherein the target antigen elicits a Th2 response. According to a further aspect, the composition comprises a target antigen conjugated to a bi-specific antibody that selectively binds both a T cell receptor and CD4, wherein the target antigen elicits a Th1 response. According to still another aspect, the composition comprises an isolated B cell contacted in vitro with a target antigen conjugated to an antibody that selectively binds a B cell cell-surface immunoglobulin, wherein the target antigen elicits a Th2 response, and a pharmaceutically acceptable carrier. According to a further aspect, the composition comprises an isolated CD4⁺ helper T cell contacted in vitro with a target antigen conjugated to an antibody that selectively binds a T cell receptor, wherein the target antigen elicits a Th2 response, and a pharmaceutically acceptable carrier. According to yet another aspect, the composition comprises an isolated CD4⁺ helper T cell contacted in vitro with a target antigen conjugated to a bi-specific antibody that selectively binds both a T cell receptor and CD4, wherein the target antigen elicits a Th1 response, and a pharmaceutically acceptable carrier.

These and other aspects of the invention are described in greater detail below. Each of the limitations of the invention can encompass various embodiments of the invention. It is, therefore, anticipated that each of the limitations of the invention, involving any one element or combinations of elements can be included in each aspect of the invention.

### DETAILED DESCRIPTION OF THE INVENTION

The invention, in one important part, provides methods and compositions for promoting antigen-specific immune responses with an appropriate cytokine bias. More specifically, the present invention relates to methods of using autologous adoptive immunotherapy with antigen-specific, ex vivo-primed T cells or B cells to promote antigen-specific immune responses with Th1 or Th2 cytokine bias. By “ex vivo” it is meant that cells have been isolated from a subject, are temporarily cultured and manipulated in vitro, and returned to the subject. As used herein, a subject is a human, non-human primate, cow, horse, pig, sheep, goat, dog, cat or rodent.

It has been discovered that the ex vivo treatment of lymphocytes present in a relatively small volume of a blood sample, given an appropriate antigenic stimulation, causes the lymphocytes to develop properties similar to those present in very efficient APC. When reinfused into the subject the manipulated lymphocytes are a significant priming source for memory cells. Additionally, a directed response to a specific cytokine bias (Th1 or Th2) can be established, depending upon the antigenic stimulation. This is in contrast to a direct, in vivo administration of an antigen which frequently results in the generation of only few antigen-specific loaded APCs and Th1 as a “default” response. It has been discovered, that a direct, in vivo administration of an antigen leaves a large number of APCs which present antigen through MHC Class II, void of antigen (i.e., with their MHC Class II pockets unloaded or empty). As a result, APCs with empty MHC Class II pockets die upon antigenic stimulation of T cells through MHC Class II-mediated cell-death. It has been discovered, that loading of MHC Class II pockets with antigen in vitro, prevents MHC Class II-mediated cell-death of these cells, and makes them efficient APCs.

The present invention thus becomes useful in a wide range of applications, including pre-exposure vaccination of individuals with both in vivo and in vitro primed T cells, treatment of cancer subjects using tumor-targeted T cell immunotherapy, treatment of bone marrow transplant subjects (for whom opportunistic infections, such as CMV, are problematic and yet amenable to treatment with targeted T cells such as CMV-targeted cytotoxic lymphocytes), enhancement of conventional vaccination efficacy through T cell adjuvant therapy, treatment of outbreaks of emergent or re-emergent pathogens, etc.

According to one aspect of the invention, a method of autologous adoptive immunotherapy, is provided. The method involves obtaining B cells that have been originally isolated from a subject and subsequently contacted in vitro with a target antigen-conjugate to produce target antigen manipulated B cells, and infusing the target antigen manipulated B cells into the subject. The target antigen conjugate, comprises a target antigen that elicits a Th2 response, conjugated to an antibody that selectively binds a B cell cell-surface immunoglobulin. In important embodiments, the target antigen manipulated B cells enhance an antigen-specific immune cell response to the target antigen that elicits a Th2 response in a subject.

A “target antigen that elicits a Th2 response,” as used herein, is a molecule capable of provoking an immune response associated with the induction of Th2 cytokines. Typically, in a Th2 response at least interleukins IL-4, IL-5, IL-6, IL-9, IL-10, and/or IL-13 are produced (Mosmann, 1989, Annu. Rev. Immunol., 7:145-173). The term “Th2 antigen” broadly includes any type of molecule which is recognized by a host immune system as being foreign and results in the induction of Th2 cytokines. Antigens that elicit a Th2 response include, but are not limited to peptides, polypeptides, cells, cell extracts, polysaccharides, polysaccharide conjugates, lipids, glycolipids and carbohydrates, cancer antigens, microbial antigens, and allergans. Antigens may be given in a crude, purified or recombinant form, and polypeptide/peptide antigens, including peptide mimics of polysaccharides, may also be encoded within nucleic acids. Sources of such antigens include, but are not limited to, extracellular parasites (e.g., Entamoeba Histolytica, Giardia Lamblia, hookworms and other helminths, etc.), Cornebacterium Diptheriae, Vibrio Cholerae, Neisseria Meningitidis, and Staphylococcus Aureus.

“Cytokine” is a generic term for nonantibody soluble proteins which are released from one cell subpopulation and which act as intercellular mediators, for example, in the generation or regulation of an immune response. See Human Cytokines: Handbookfor Basic & Clinical Research (Aggrawal, et al. eds., Blackwell Scientific, Boston, Mass. 1991) (which is hereby incorporated by reference in its entirety for all purposes). Cytokines include, e.g., interleukins IL-1 through IL-15, tumor necrosis factors α & β, interferons (IFNs) α, β, and γ, tumor growth factor beta (TGF-β), colony stimulating factor (CSF) and granulocyte monocyte colony stimulating factor (GM-CSF). The action of each cytokine on its target cell is mediated through binding to a cell surface receptor. Cytokines share many properties with hormones, but are distinct from classical hormones in that in vivo, they generally act locally on neighboring cells within a tissue. The activities of cytokines range from promoting cell growth (e.g., IL-2, IL-4, and IL-7), and arresting growth (IL-10, tumor necrosis factor and TGF-β), to inducing viral resistance (IFNs α, β, and γ). See Fundamental Immunology (Paul ed., Raven Press, 2nd ed. 1989); Encyclopedia of Immunology, (Roitt ed., Academic Press 1992) (which are hereby incorporated by reference in their entirety for all purposes). A number of in vitro methods known in the art can be utilized to establish whether an antigen gives rise to a Th1 or Th2 response. Commercial ELISA based assays for any of the Th1 or Th2-specific cytokines are readily available (e.g. Promega, Madison, Wis.), and can be used according to the manufacturer's protocols.

As used herein, with respect to the lymphocytes of the invention (B cells and T cells), “isolated” means separated from its native environment and present in sufficient quantity and purity to permit its identification or use. Isolated cells may, but need not be, substantially pure. In certain embodiments of the present invention the isolated cells are substantially pure cells. The term “substantially pure” means that the specific cell population (B cell or T cell) is essentially free of other cells or substances with which it (they) may be found in nature or in vitro systems, to an extent practical and appropriate for their intended use. In preferred embodiments, the isolated B cells of the invention are mature B cells and in some embodiments free of other immature cells of the B cell lineage. Isolated CD4⁺ helper T cells of the invention are by definition mature cells, and are free of other mature (e.g., CD8⁺) and immature cells of the T cell lineage. Substantially pure lymphocytes according to the invention may be produced by techniques well known in the art.

An isolated CD4⁺ helper T cell as used herein is a cell within a lymphocyte population that is enriched for CD4⁺ helper T cells by selectively eliminating B cells and other lymphocytes present in the PBMC sample, although a small number of B cells and other lymphocytes may be present. Methods for CD4⁺ helper T cell enrichment are described in more detail below (see under isolation of peripheral blood or monocytes). Various embodiments are provided, wherein the CD4⁺ helper T cell-enriched lymphocyte population contains at least 50%, at least 75%, at least 90%, or at least 95% T cells. Similarly, an isolated B cell as used herein is a mature B cell within a lymphocyte population that is enriched for mature B cells by selectively eliminating T cells and other lymphocytes present in the PBMC sample, although a small number of T cells and other lymphocytes may be present. Methods for mature B cell enrichment are described in more detail below (see under isolation of peripheral blood or monocytes). Various embodiments are provided, wherein the mature B cell-enriched lymphocyte population contains at least 50%, at least 75%, at least 90%, or at least 95% T cells.

According to the invention, the target antigen conjugate comprises a target antigen conjugated to an antibody, fragments of antibodies, peptides, and/or other molecules having the ability to selectively bind to: (i) a B cell cell-surface immunoglobulin, (ii) CD4 on the surface of a T cell, and/or (iii) both CD4 and T cell receptor on the surface of the same T cell, depending upon the cytokine bias response required (Th1 or Th2). For example, if a response to a Th1 antigen is desired, a target antigen conjugate comprising the Th1 target antigen conjugated to an antibody, fragments of antibodies, peptides, and/or other molecules having the ability to selectively bind to both CD4 and T cell receptor on the surface of the same CD4⁺ helper T cell, would be processed in vitro according to the invention (i.e. contacted with an isolated naive CD4⁺ helper T cell from a subject). If a response to a Th2 antigen is desired, a target antigen conjugate comprising the Th2 target antigen conjugated to an antibody, fragments of antibodies, peptides, and/or other molecules having the ability to selectively bind to a B cell cell-surface immunoglobulin, or to CD4 on the surface of a T cell, would be processed in vitro according to the invention (i.e. contacted with an isolated naive mature B cell, or contacted with an isolated naive CD4⁺ helper T cell, from a subject, respectively). Antibodies include polyclonal and monoclonal antibodies, prepared according to conventional methodology.

Antibodies useful according to this aspect of the invention are available from a number of commercial sources. For example, anti-human-immunoglobulin antibodies can be purchased at least from Southern Biotechnology Associates, Inc., Birmingham, AL, and include, at least Goat Anti-Human Ig(H+L); Goat F(ab′)₂ Anti-Human Ig(H+L); Goat Anti-Human IgM; Goat F(ab′)₂ Anti-Human IgM; Goat Anti-Human IgD; Goat F(ab′)₂ Anti-Human IgD; Goat Anti-Human IgG; Goat F(ab′)₂ Anti-Human IgG; Goat Anti-Human IgA; Goat F(ab′)₂ Anti-Human IgA; Goat Anti-Human kappa; Goat F(ab′)₂ Anti-Human kappa; Goat Anti-Human lambda; Goat F(ab′)₂ Anti-Human lambda; Mouse Anti-Human IgM; Mouse Anti-Human IgD; Mouse Anti-Human IgG; Mouse Anti-Human IgG₁; Mouse Anti-Human IgG₂; Mouse Anti-Human IgA₁; Mouse Anti-Human IgA₂; Mouse Anti-Human IgE; Mouse Anti-Human lambda; Mouse Anti-Human IgG₄; Mouse Anti-Human IgG₃; Mouse Anti-Human kappa; and Mouse Anti-Human IgE.

Anti-human CD4 antibodies can also be purchased at least from Southern Biotechnology Associates, Inc., including cat. no. 9521-01 for a .Mouse Anti-Human CD4 antibody, or from Ancell, Inc., Bayport, Minn., including cat. no. 147-020 for a Mouse Anti-Human CD4 (Domain 1) and cat. no. 148-020 for a Mouse Anti-Human CD4 (Domain 2) antibody.

Anti-human T cell receptor (TCR) antibodies can be purchased at least from Serotec, Inc., Raleigh, N.C., including antibodies against TCR alpha/beta—alpha chain (framework); TCR alpha/beta—beta chain (framework); TCR alpha/beta—Pan (framework); TCR gamma/delta—gamma chain (framework); TCR gamma/delta—delta chain (framework); TCR gamma/delta—delta chain (variable); TCR V alpha 2; TCR V alpha 12.1; TCR V alpha 24; TCR V beta 1; TCR V beta 2; TCR V beta 3; TCR V beta 3.1; TCR V beta 5.1; TCR V beta 5.2; TCR V beta 5.2, 5.3; TCR V beta 5.3; TCR V beta 6.7; TCR V beta 7; TCR V beta 7.1; TCR V beta 8; TCR V beta 9; TCR V beta 11.1; TCR V beta 12; TCR V beta 12.1; TCR V beta 13.1; TCR V beta 13.1, 13.3; TCR V beta 13.6; TCR V beta 14; TCR V beta 16; TCR V beta 17; TCR V beta 18; TCR V beta 20; TCR V beta 21.3; TCR V beta 22; TCR V beta 23; TCR V gamma 1; TCR V gamma 2; TCR V gamma 4; TCR V gamma 9; TCR V delta 1; TCR V delta 2; TCR V delta 3; TCR zeta chain; or from Ancell, Inc., Bayport, Minn., including cat. no. 101-020 for a Mouse Anti-Human TCR C beta 1 antibody, and cat. no. 102-020 for a Mouse Anti-Human TCR V beta 3 antibody.

In some aspects of the invention the target antigen is conjugated to a lymphocyte cell-surface binding peptide or molecule. The binding peptides or molecules can be delivered directly to the cell to act on the lymphocyte cell-surface. As long as they are delivered by a mechanism which will not facilitate uptake of the molecule into the cell, then the lymphocyte cell-surface binding peptide or molecule will be targeted to the lymphocyte cell-surface. The lymphocyte cell-surface binding peptide or molecule may also be attached to a targeting molecule which targets the peptide or molecule to the cell of interest, as discussed in more detail below.

The lymphocyte cell-surface binding peptides and molecules of the invention can be identified using routine assays, such as the binding and activation assays described in the Examples and elsewhere throughout this patent application.

The binding peptides of the invention are isolated peptides. As used herein, with respect to peptides, the term “isolated peptides” means that the peptides are substantially pure and are essentially free of other substances with which they may be found in nature or in vivo systems to an extent practical and appropriate for their intended use. In particular, the peptides are sufficiently pure and are sufficiently free from other biological constituents of their hosts cells so as to be useful in, for example, producing pharmaceutical preparations or sequencing. Because an isolated peptide of the invention may be admixed with a pharmaceutically acceptable carrier in a pharmaceutical preparation, the peptide may comprise only a small percentage by weight of the preparation. The peptide is nonetheless substantially pure in that it has been substantially separated from the substances with which it may be associated in living systems.

The binding peptides also may easily be synthesized or produced by recombinant means by those of skill in the art. Methods for preparing or identifying peptides which bind to a particular target are well known in the art. Molecular imprinting, for instance, may be used for the de novo construction of macromolecular structures such as peptides which bind to a particular molecule. See, for example, Kenneth J. Shea: “Molecular Imprinting of Synthetic Network Polymers: The De Novo synthesis of Macromolecular Binding and Catalytic Sites,” TRIP Vol. 2, No. 5, May 1994; Klaus Mosbach: “Molecular Imprinting,” Trends in Biochem. Sci., 19(9) January 1994; and Wulff, G.: “Polymeric Reagents and Catalysts” (Ford, W. T., Ed.) ACS Symposium Series No. 308, pp 186-230, American Chemical Society (1986). One method for preparing mimics of the known binding peptides involves the steps of: (i) polymerization of functional monomers around a known binding peptide or the binding region of an antibody which also binds to the targets (the template) that exhibits a desired activity; (ii) removal of the template molecule; and then (iii) polymerization of a second class of monomers in the void left by the template, to provide a new molecule which exhibits one or more desired properties which are similar to that of the template. In addition to preparing peptides in this manner other binding molecules which have the same function as the binding peptides useful according to the invention such as polysaccharides, nucleosides, drugs, nucleoproteins, lipoproteins, carbohydrates, glycoproteins, steroids, lipids, and other biologically active materials can also be prepared. This method is useful for designing a wide variety of biological mimics that are more stable than their natural counterparts, because they are typically prepared by the free radical polymerization of functional monomers, resulting in a compound with a nonbiodegradable backbone. Other methods for designing such molecules include for example drug design based on structure activity relationships which require the synthesis and evaluation of a number of compounds and molecular modeling.

The binding peptides may also be identified by conventional screening methods such as phage display procedures (e.g., methods described in Hart, et al., J. Biol. Chem. 269:12468 (1994)). Hart et al. report a filamentous phage display library for identifying novel peptide ligands for mammalian cell receptors. In general, phage display libraries using, e.g., M13 or fd phage, are prepared using conventional procedures such as those described in the foregoing reference. The libraries display inserts containing from 4 to 80 amino acid residues. The inserts optionally represent a completely degenerate or a biased array of peptides. Ligands having the appropriate binding properties are obtained by selecting those phages which express on their surface a ligand that binds to the target molecule. These phages then are subjected to several cycles of reselection to identify the peptide ligand-expressing phages that have the most useful binding characteristics. Typically, phages that exhibit the best binding characteristics (e.g., highest affinity) are further characterized by nucleic acid analysis to identify the particular amino acid sequences of the peptides expressed on the phage surface and the optimum length of the expressed peptide to achieve optimum binding. Alternatively, such peptide ligands can be selected from combinatorial libraries of peptides containing one or more amino acids. Such libraries can further be synthesized which contain non-peptide synthetic moieties which are less subject to enzymatic degradation compared to their naturally-occurring counterparts.

To determine whether a peptide binds to the appropriate target any known binding assay may be employed. For example, in the case of a peptide that binds to the lymphocyte cell-surface, the peptide may be immobilized on a surface and then contacted with a labeled lymphocyte cell-surface molecule (or vice versa). The amount of lymphocyte cell-surface molecule which interacts with the peptide or the amount which does not bind to the peptide may then be quantitated to determine whether the peptide binds to a lymphocyte cell-surface molecule. A surface having a known peptide that binds to a lymphocyte cell-surface molecule such as a commercially available monoclonal antibody immobilized thereto may serve as a positive control. Several types of anti-B cell and anti-T cell antibodies are commercially available from Santa Cruz Biotechnology, Inc., Pharmingen, Inc., and other companies.

Screening of peptides of the invention, also can be carried out utilizing a competition assay. If the peptide being tested competes with the known monoclonal antibody, as shown by a decrease in binding of the known monoclonal antibody, then it is likely that the peptide and the known monoclonal antibody bind to the same, or a closely related, epitope. Still another way to determine whether a peptide has the specificity of the known monoclonal antibody is to pre-incubate the known monoclonal antibody with the target with which it is normally reactive, and then add the peptide being tested to determine if the peptide being tested is inhibited in its ability to bind the target. If the peptide being tested is inhibited then, in all likelihood, it has the same, or a functionally equivalent, epitope and specificity as the known monoclonal antibody.

By using the known anti-lymphocyte cell-surface (and other target) monoclonal antibodies of the invention, it is also possible to produce anti-idiotypic antibodies which can be used to screen other antibodies to identify whether the antibody has the same binding specificity as the known monoclonal antibody. Such anti-idiotypic antibodies can be produced using well-known hybridoma techniques (Kohler and Milstein, Nature, 256:495, 1975). An anti-idiotypic antibody is an antibody which recognizes unique determinants present on the known monoclonal antibodies. These determinants are located in the hypervariable region of the antibody. It is this region which binds to a given epitope and, thus, is responsible for the specificity of the antibody. An anti-idiotypic antibody can be prepared by immunizing an animal with the known monoclonal antibodies. The immunized animal will recognize and respond to the idiotypic determinants of the immunizing known monoclonal antibodies and produce an antibody to these idiotypic determinants. By using the anti-idiotypic antibodies of the immunized animal, which are specific for the known monoclonal antibodies of the invention, it is possible to identify other clones with the same idiotype as the known monoclonal antibody used for immunization. Idiotypic identity between monoclonal antibodies of two cell lines demonstrates that the two monoclonal antibodies are the same with respect to their recognition of the same epitopic determinant. Thus, by using anti-idiotypic antibodies, it is possible to identify other hybridomas expressing monoclonal antibodies having the same epitopic specificity.

It is also possible to use the anti-idiotype technology to produce monoclonal antibodies which mimic an epitope. For example, an anti-idiotypic monoclonal antibody made to a first monoclonal antibody will have a binding domain in the hypervariable region which is the image of the epitope bound by the first monoclonal antibody.

In one embodiment the binding peptides useful according to the invention are antibodies or functionally active antibody fragments. Antibodies are well known to those of ordinary skill in the science of immunology. Many of the binding peptides described herein are available from commercial sources as intact functional antibodies. As used herein, the term “antibody” means not only intact antibody molecules but also fragments of antibody molecules retaining specific binding ability. Such fragments are also well known in the art and are regularly employed both in vitro and in vivo. In particular, as used herein, the term “antibody” means not only intact immunoglobulin molecules but also the well-known active fragments F(ab′)2, and Fab. F(ab′)2, and Fab fragments which lack the Fc fragment of intact antibody, clear more rapidly from the circulation, and may have less non-specific tissue binding of an intact antibody (Wahl et al., J. Nucl. Med. 24:316-325 (1983)).

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FRI through FR4) separated respectively by three complementarity determining regions (CDR1 through CDR3). The CDRs, and in particular the CDR3 regions, and more particularly the heavy chain CDR3, are largely responsible for antibody specificity.

It is now well-established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. Thus, for example, PCT International Publication Number WO 92/04381 teaches the production and use of humanized murine RSV antibodies in which at least a portion of the murine FR regions have been replaced by FR regions of human origin. Such antibodies, including fragments of intact antibodies with antigen-binding ability, are often referred to as “chimeric” antibodies. A “humanized monoclonal antibody” as used herein is a human monoclonal antibody or functionally active fragment thereof having human constant regions and a binding CDR3 region from a mammal of a species other than a human. Humanized monoclonal antibodies may be made by any method known in the art. Humanized monoclonal antibodies, for example, may be constructed by replacing the non-CDR regions of a non-human mammalian antibody with similar regions of human antibodies while retaining the epitopic specificity of the original antibody. For example, non-human CDRs and optionally some of the framework regions may be covalently joined to human FR and/or Fc/pFc′ regions to produce a functional antibody. There are entities in the United States which will synthesize humanized antibodies from specific murine antibody regions commercially, such as Protein Design Labs (Mountain View Calif.). For instance, a humanized form of the Pharmingen anti-Fas antibody used in the attached Examples could be easily prepared and used according to the methods of the invention.

European Patent Application 0239400, the entire contents of which is hereby incorporated by reference, provides an exemplary teaching of the production and use of humanized monoclonal antibodies in which at least the CDR portion of a murine (or other non-human mammal) antibody is included in the humanized antibody. Briefly, the following methods are useful for constructing a humanized CDR monoclonal antibody including at least a portion of a mouse CDR. A first replicable expression vector including a suitable promoter operably linked to a DNA sequence encoding at least a variable domain of an Ig heavy or light chain and the variable domain comprising framework regions from a human antibody and a CDR region of a murine antibody is prepared. Optionally a second replicable expression vector is prepared which includes a suitable promoter operably linked to a DNA sequence encoding at least the variable domain of a complementary human Ig light or heavy chain respectively. A cell line is then transformed with the vectors. Preferably the cell line is an immortalized mammalian cell line of lymphoid origin, such as a myeloma, hybridoma, trioma, or quadroma cell line, or is a normal lymphoid cell which has been immortalized by transformation with a virus. The transformed cell line is then cultured under conditions known to those of skill in the art to produce the humanized antibody.

As set forth in European Patent Application 0239400 several techniques are well known in the art for creating the particular antibody domains to be inserted into the replicable vector. (Preferred vectors and recombinant techniques are discussed in greater detail below.) For example, the DNA sequence encoding the domain may be prepared by oligonucleotide synthesis. Alternatively a synthetic gene lacking the CDR regions in which four framework regions are fused together with suitable restriction sites at the junctions, such that double stranded synthetic or restricted subcloned CDR cassettes with sticky ends could be ligated at the junctions of the framework regions. Another method involves the preparation of the DNA sequence encoding the variable CDR containing domain by oligonucleotide site-directed mutagenesis. Each of these methods is well known in the art. Therefore, those skilled in the art may construct humanized antibodies containing a murine CDR region without destroying the specificity of the antibody for its epitope.

Human monoclonal antibodies may be made by any of the methods known in the art, such as those disclosed in U.S. Pat. No. 5,567,610, issued to Borrebaeck et al., U.S. Pat. No. 565,354, issued to Ostberg, U.S. Pat. No. 5,571,893, issued to Baker et al, Kozber, J. Immunol. 133: 3001 (1984), Brodeur, et al., Monoclonal Antibody Production Techniques and Applications, p. 51-63 (Marcel Dekker, Inc, New York, 1987), and Boerner el al., J. Immunol., 147: 86-95 (1991). In addition to the conventional methods for preparing human monoclonal antibodies, such antibodies may also be prepared by immunizing transgenic animals that are capable of producing human antibodies (e.g., Jakobovits et al., PNAS USA, 90: 2551 (1993), Jakobovits et al., Nature, 362: 255-258 (1993), Bruggermann et al., Year in Immuno., 7:33 (1993) and U.S. Pat. No. 5,569,825 issued to Lonberg).

The binding peptides may also be functionally active antibody fragments. Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The pFc′ and Fc regions of the antibody, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. An isolated F(ab′)2 fragment is referred to as a bivalent monoclonal fragment because of its two antigen binding sites. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd (heavy chain variable region). The Fd fragments are the major determinant of antibody specificity (a single Fd fragment may be associated with up to ten different light chains without altering antibody specificity) and Fd fragments retain epitope-binding ability in isolation.

The terms Fab, Fc, pFc′, F(ab′)2 and Fv are used consistently with their standard immunological meanings [Klein, Immunology (John Wiley, New York, N.Y., 1982); Clark, W. R. (1986) The Experimental Foundations of Modern Immunology (Wiley & Sons, Inc., New York); Roitt, I. (1991) Essential Immunology, 7th Ed., (Blackwell Scientific Publications, Oxford)].

Thus, as will be apparent to one of ordinary skill in the art, the antibody conjugates of the invention also include F(ab′)₂, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

According to the invention, a target antigen is conjugated to an antibody (see foregoing description). Such conjugation/linking is well known and commonly used in the art, for example, by utilization of biotin/avidin (streptavidin) bonds, etc. The chemistry for attaching linker moieties for connecting two agents is also well known and commonly used in the art (for a review see Wong S S, et al., 1992, Enzyme Microb Technol, 14:866-874). Avidin-Biotin chemistry provides an efficient, easy use and cost-effective way for the attachment of target antigens of the invention to antibodies. A wide assortment of insolubilized derivatives of avidin and streptavidin are available commercially (Avidin-Biotin Chemistry: A Handbook—Developed by Pierce Technical Assistance experts).

In general, the target moiety (antigen) is coupled to an antibody, fragments of antibodies, peptides, and/or other molecules (collectively “antibody”), having the ability to selectively bind molecules in the surface of mature lymphocytes. The molecules may be directly coupled to one another, such as by conjugation or may be indirectly coupled to one another. If the molecules are linked to one another, then the targeting moiety is covalently or noncovalently bound to the “antibody” in a manner that preserves the targeting specificity of the “antibody”. As used herein, “linked” or “linkage” means two entities are bound to one another by any physiochemical means. It is important that the linkage be of such a nature that it does not impair substantially the effectiveness of the “antibody” or the binding/presenting specificity of the target moiety. Keeping these parameters in mind, any linkage known to those of ordinary skill in the art may be employed, covalent or noncovalent. Such means and methods of linkage are well known to those of ordinary skill in the art (see foregoing description).

Linkage according to the invention need not be direct linkage. The components of the compositions of the invention may be provided with functionalized groups to facilitate their linkage and/or linker groups may be interposed between the components of these compositions to facilitate their linkage. In addition, the components of the present invention may be synthesized in a single process, whereby the components could be regarded as one in the same entity. For example, a target moiety specific for a tumor cell could be synthesized together with the “antibody.” These and other modifications are intended to be embraced by the present invention.

Specific examples of covalent bonds include those wherein bifunctional cross-linker molecules are used. The cross-linker molecules may be homobifunctional or heterobifunctional, depending upon the nature of the molecules to be conjugated. Homobifunctional cross-linkers have two identical reactive groups. Heterobifunctional cross-linkers have two different reactive groups that allow sequential conjugation reaction. Various types of commercially available cross-linkers are reactive with one or more of the following groups: primary amines, secondary amines, sulfhydriles, carboxyls, carbonyls and carbohydrates.

Non-covalent methods of conjugation also may be used to join the target moiety and the “antibody.” Non-covalent conjugation may be accomplished by direct or indirect means including hydrophobic interaction, ionic interaction, intercalation, binding to major or minor grooves of a nucleic acid and other affinity interactions.

Covalent linkages may be noncleavable in physiological environments or cleavable in physiological environments, such as linkers containing disulfide bonds. Such molecules may resist degradation and/or may be subject to different intracellular transport mechanisms. One of ordinary skill in the art will be able to ascertain without undue experimentation the preferred bond for linking the target moiety and the “antibody,” based on the chemical properties of the molecules being linked and the preferred characteristics of the bond.

According to one aspect of the invention the contacting of isolated B cells to a target antigen conjugated to an antibody that selectively binds a B cell cell-surface immunoglobulin, occurs in vitro, under conditions sufficient to produce target antigen manipulated B cells. “Target antigen manipulated B cells,” as used herein, is meant to describe isolated B cells that express on their surface MHC Class II molecules loaded with target antigen, suitable for presentation to, for example, CD4⁺ helper T cells that will eventually provide the necessary signals for the expansion of long-lived memory T cells. Activation of the isolated B cells in vitro with a target antigen conjugated to an antibody that selectively binds a B cell cell-surface immunoglobulin, results in the endocytosis of the target antigen by the cell, followed by its transport from early endosomes into late endosomes or pre-lysosomes, where it is fragmented, and where peptide resulting from it is loaded into MHC Class II molecules. The MHC Class II molecules have been transported from the rough endoplasmic reticulum (RER), through the Golgi apparatus, to the peptide-containing vesicles. MHC Class II molecules loaded with target antigen are then transported to the surface of the cell. By “conditions sufficient” to produce target antigen manipulated B cells is therefore meant to describe conditions that will allow for the foregoing processing of target antigen to occur, resulting in the target antigen peptide's final presentation on the surface of the cell in conjunction with MHC Class II molecules. Such conditions also include culture conditions which in turn refer collectively to a combination of conditions known in the art (e.g., temperature, CO₂ and O₂ content, nutritive media, type of culture vessel, time-length, cytokines present, etc.), that can be easily optimized by a person skilled in the art according to the above-identified needs. Typically, an overnight culture of either the B cell-enriched or T cell-enriched cell populations originally contained in a 10 ml sample of a subject's peripheral blood, cultured at 37° C., 5% CO₂, optionally cytokine-free, together with the preferred target antigen conjugate, is sufficient to allow processing of the target antigen and its loading onto the MHC class II molecule for presentation.

A number of ways exist in the art for one of ordinary skill to establish whether target antigen manipulated B cells (or T cells) have been obtained. One direct assay is to detect the antigen/MHC class II complex on the surface of the cell with a specific antibody. An indirect, functional assay is by stimulation of T cells in the presence of target antigen manipulated B cells (or T cells) according to the invention. Such stimulation induces T cell receptor gene rearrangement and an antigen specific T cell response that can be measured using a proliferation assay (see Examples), or just by measuring IL-2 production (using assays well known in the art such as radio-active assays or commercially available non-radioactive, ELISA based assays, from e.g. Promega, Madison, Wis.). Additional methods could easily be established by a person of ordinary skill in the art, without undue experimentation (see also Sprent J, et al., J. Immunother, 1998, 21(3):181-187; Berridge M J, Crit Rev Immunol, 1997, 17(2):155-178; Owen M J, et al., Curr Opin Immunol, 1996, 8(2):191-198; Whitfield J F, et al., Mol Cell Biochem, 1979, 27(3):155-179; Fauci A S, et al., Ann Intern Med, 1983, 99(1):61-75).

According to some embodiments of the invention, the B cells can be isolated from peripheral blood, or from an in vitro hematopoietic progenitor cell culture. Peripheral blood containing mononuclear cells can be isolated from a subject (using known methods, e.g., apheresis), and a portion of the sample is mixed with an anticoagulant, e.g., heparin, sodium citrate, ethylenediaminetetraacetic acid, sodium oxalate. The blood-anticoagulant mixture then is diluted in a physiologically acceptable solution such as sodium chloride or phosphate buffered solution. Mononuclear cells are recovered by layering the blood-anticoagulant composition onto a centrifugation separation medium such as Ficoll-Hypaque (Pharmacia Corporation) or Lymphocyte Separation Medium (Litton Bionetics Corporation). The layered mixture then is centrifuged, and the interface containing the mononuclear cells is collected and washed. The concentration of mononuclear cells can be in the range of about 0.05-5.0×10⁶ cells/ml of blood collected. Although any standard tissue culture medium can be utilized in the process of this invention, the cells are preferably cultured in a complete medium consisting of RPMI 1640 (Gibco-BRL, Grand Island, N.Y.), supplemented with 2 mM L-Glutamine, streptomycin (100 mg/ml), penicillin (100 U/ml, and 5% heat-inactivated autologous plasma. Enriched monocyte preparations can be prepared by resetting of PMBCs with AET-treated sheep red blood cells and removal of E-rosetting cells on Ficoll-Hypaque density gradients, followed by cold aggregation of monocytes as essentially described in Zupo et al. (Eur. J. Immunol., 1991, 21:351). Mature B cells and CD4⁺ helper T cells may be further purified from the PMBC preparations by depletion of monocytes, and NK cells using Lympho-Kwik T and/or Lympho-Kwik B (One Lambda, Los Angeles, Calif.), according to the manufacturer's protocol.

The hematopoietic progenitor cells, which are also isolated from the subject, are useful according to the invention. These cells can be cultured in vitro in a variety of cultured vessels, preferably together with an exogenously added agent that includes stromal cell conditioned medium and/or a hematopoietic growth factor that promotes hematopoietic cell maintenance, expansion and/or differentiation, and influences cell localization, to yield the more mature blood cells useful in the invention. Such differentiated cells are preferably mature B cells and T cells.

“Hematopoietic progenitor cells” as used herein refers to immature blood cells having the capacity to self-renew and to differentiate into the more mature blood cells (also described herein as “progeny”) comprising granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), and monocytes (e.g., monocytes, macrophages). It is known in the art that such cells may or may not include CD34⁺ cells. CD34⁺ cells are immature cells present in the “blood products” described below, express the CD34 cell surface marker, and are believed to include a subpopulation of cells with the “progenitor cell” properties defined above. It is well known in the art that hematopoietic progenitor cells include pluripotent stem cells, multipotent progenitor cells (e.g., a lymphoid stem cell), and/or progenitor cells committed to specific hematopoietic lineages. The progenitor cells committed to specific hematopoietic lineages may be of T cell lineage, B cell lineage, dendritic cell lineage, Langerhans cell lineage and/or lymphoid tissue-specific macrophage cell lineage.

The hematopoietic progenitor cells can be obtained from blood products. A “blood product” as used in the present invention defines a product obtained from the body or an organ of the body containing cells of hematopoietic origin. Such sources include unfractionated bone marrow, umbilical cord, peripheral blood, liver, thymus, lymph and spleen. It will be apparent to those of ordinary skill in the art that all of the aforementioned crude or unfractionated blood products can be enriched for cells having “hematopoietic progenitor cell” characteristics in a number of ways. For example, the blood product can be depleted from the more differentiated progeny. The more mature, differentiated cells can be selected against, via cell surface molecules they express. Additionally, the blood product can be fractionated selecting for CD34⁺ cells. As mentioned earlier, CD34⁺ cells are thought in the art to include a subpopulation of cells capable of self-renewal and pluripotentiality. Such selection can be accomplished using, for example, commercially available magnetic anti-CD34 beads (Dynal, Lake Success, N.Y.). Unfractionated blood products can be obtained directly from a donor or retrieved from cryopreservative storage.

A variety of “culture vessels” can be used according to the present invention. Commercially available incubation vessels include stirring flasks (Corning, Inc., Corning, N.Y.), stirred tank reactors (Verax, Lebanon, N.H.), airlift reactors, suspension cell reactors, cell adsorption reactors and cell entrapment reactors, petri dishes, multiwell plates, flasks, bags and hollow fiber devices, Cellfoam (Cytomatrix, Woburn, Mass.), maxisorb plates (NUNC), and cell culture systems (e.g., Aastrom Cell Production System, see also U.S. Pat. No. 5,635,386, entitled “Methods for regulating the specific lineages of cells produced in a human hematopoietic cell culture”, issued to Palsson et al., and U.S. Pat. No. 5,646,043, entitled “Methods for the ex vivo replication of human stem cells and/or expansion of human progenitor cells”, issued to Emerson, et al.). In general, such vessels are formed of one or more of the following components: polystyrene, polypropylene, acrylic, nylon, and glass.

It should be noted that the isolated B cells contacted in vitro with a target antigen conjugate may alternatively be cryopreserved for later use. Cryopreservation of cells cultured in vitro is well established in the art. Subsequent to isolation (and/or mitotic inactivation) of a cell sample, cells may be cryopreserved by first suspending the cells in a cryopreservation medium and then gradually freezing the cell suspension. Frozen cells are typically stored in liquid nitrogen or at an equivalent temperature in a medium containing serum and a cryopreservative such as dimethyl sulfoxide.

In certain embodiments, the in vitro contacting of the isolated B cells with a target antigen conjugate, further comprises contacting the isolated B cells with a B cell co-stimulating agent and/or a Th2 cytokine. In preferred embodiments, the B cell co-stimulating agent can be TSA-1, CD2, CD5, CD24, CD28, CD40L, CD49a, CD80, CD81 and/or CD86, and the Th2 cytokine can be IL-4, IL-5, IL-6, IL-9, IL-10, and/or IL-13.

According to another aspect of the invention, a method of autologous adoptive immunotherapy, is provided. The method involves obtaining CD4⁺ helper T cells that have been originally isolated from a subject and subsequently contacted in vitro with a target antigen-conjugate to produce target antigen manipulated CD4⁺ helper T cells, and infusing the target antigen manipulated CD4⁺ helper T cells into the subject. The target antigen conjugate, comprises a target antigen that elicits a Th2 response, conjugated to an antibody that selectively binds a T cell receptor. In important embodiments, the target antigen manipulated CD4⁺ helper T cells enhance an antigen-specific immune cell response to the target antigen that elicits a Th2 response in a subject. Target antigens that elicit a Th2 response are as described above.

According to a further aspect of the invention, a method of autologous adoptive immunotherapy, is provided. The method involves obtaining CD4⁺ helper T cells that have been originally isolated from a subject and subsequently contacted in vitro with a target antigen-conjugate to produce target antigen manipulated CD4⁺ helper T cells, and infusing the target antigen manipulated CD4⁺ helper T cells into the subject. The target antigen conjugate, comprises a target antigen that elicits a Th1 response, conjugated to a bi-specific antibody that selectively binds both a T cell receptor and CD4 on the same CD4⁺ helper T cells. In important embodiments, the target antigen manipulated CD4⁺ helper T cells enhance an antigen-specific immune cell response to the target antigen that elicits a Th1 response in a subject.

A “target antigen that elicits a Th1 response,” as used herein, is a molecule capable of provoking an immune response associated with the induction of Th1 cytokines. The term Th1 antigen broadly includes any type of molecule which is recognized by a host immune system as being foreign and results in the induction of Th1 cytokines. Typically, in a Th1 response at least IL-2, TNF-α, and/or IFN-γ cytokines are produced. Antigens include but are not limited to peptides, polypeptides, cells, cell extracts, polysaccharides, polysaccharide conjugates, lipids, glycolipids and carbohydrates, cancer antigens, microbial antigens, and allergans. Antigens may be given in a crude, purified or recombinant form, and polypeptide/peptide antigens, including peptide mimics of polysaccharides, may also be encoded within nucleic acids. Sources of such antigens include, but are not limited to, mycobacteria (e.g., tubercolosis, leprae), intracellular pathogens (protozoans: Leishmania, Toxoplasma, Trypanosoma, Plasmodium), Schistosoma, Trichiella Spiralis, Salmonella Typhimurium, HIV, Hepatitis C, Haemophilus Influenza, antigens presented by hepatic non-parenchymal cells (e.g., Kupfer cells), OVA-conjugates, antigens that cause contact hypersensitivity (e.g., nickel, arylates, poison ivy, poison oak), allergans (e.g., birch pollen).

According to the present invention a “mature” T cell is a fully functional T cell, i.e. it has rearranged its T cell receptor and possesses the ability to exit the thymus or is found in the periphery. Examples of mature T cells include cells of a CD8^{lo}TCR⁺, a CD4^{lo}CD8^{hi}TCR⁺, and/or a CD4^{hi}TCR⁺ phenotype. A preferred mature T cell according to the invention is CD4^{hi}TCR⁺ (CD4⁺ helper T cell). A number of various other mature T cell phenotypes exist and the skilled artisan would be able to distinguish them from an immature cell and from the preferred mature T cell of the invention, using phenotypic characteristics as well as functional assays well known in the art. Examples of immature cells include cells of a CD4⁺CD8⁺TCR^{lo}, CD117⁺TCR^{lo}, etc., phenotype. Various embodiments are provided, wherein the in vitro contacting of the isolated CD4⁺ helper T cells with a target antigen conjugate, further comprises contacting the isolated CD4⁺ helper T cells with a Th2 cytokine. In preferred embodiments, the Th2 cytokine can be at least IL-4, IL-5, IL-6, IL-9, IL-10, and/or IL-13.

As described above, antigens that can be used in accordance with the methods of the invention include antigens characteristic of pathogens and cancer antigens.

Antigens that are characteristic of tumor antigens typically will be derived from the cell surface, cytoplasm, nucleus, organelles and the like of cells of tumor tissue. Examples include antigens characteristic of tumor proteins, including proteins encoded by mutated oncogenes; viral proteins associated with tumors; and tumor mucins and glycolipids. Tumors include, but are not limited to, those from the following sites of cancer and types of cancer: lip, nasopharynx, pharynx and oral cavity, esophagus, stomach, colon, rectum, liver, gall bladder, biliary tree, pancreas, larynx, lung and bronchus, melanoma of skin, breast, cervix, uteri, uterus, ovary, bladder, kidney, brain and other parts of the nervous system, thyroid, prostate, testes, Hodgkin's disease, non-Hodgkin's lymphoma, multiple myeloma and leukemia. Viral proteins associated with tumors would be those from the classes of viruses noted above. Antigens characteristic of tumors may be proteins not usually expressed by a tumor precursor cell, or may be a protein which is normally expressed in a tumor precursor cell, but having a mutation characteristic of a tumor. An antigen characteristic of a tumor may be a mutant variant of the normal protein having an altered activity or subcellular distribution. Mutations of genes giving rise to tumor antigens, in addition to those specified above, may be in the coding region, 5′ or 3′ noncoding regions, or introns of a gene, and may be the result of point mutations, frameshifts, deletions, additions, duplications, chromosomal rearrangements and the like. One of ordinary skill in the art is familiar with the broad variety of alterations to normal gene structure and expression which gives rise to tumor antigens. Specific examples of tumor antigens include: proteins such as Ig-idiotype of B cell lymphoma, mutant cyclin-dependent kinase 4 of melanoma, Pmel-17 (gp 100) of melanoma, MART-1 (Melan-A) of melanoma, p15 protein of melanoma, tyrosinase of melanoma, MAGE 1, 2 and 3 of melanoma, thyroid medullary, small cell lung cancer, colon and/or bronchial squamous cell cancer, BAGE of bladder, melanoma, breast, and squamous cell carcinoma, gp75 of melanoma, oncofetal antigen of melanoma; carbohydrate/lipids such as mucl mucin of breast, pancreas, and ovarian cancer, GM2 and GD2 gangliosides of melanoma; oncogenes such as mutant p53 of carcinoma, mutant ras of colon cancer and HER-2/neu proto-oncogene of breast carcinoma; viral products such as human papilloma virus proteins of squamous cell cancers of cervix and esophagus. It is also contemplated that proteinaceous tumor antigens may be presented by HLA molecules as specific peptides derived from the whole protein. Metabolic processing of proteins to yield antigenic peptides is well known in the art; for example see U.S. Pat. No. 5,342,774 (Boon et al.).

Preferred tumor antigens of the invention include the Melonoma tumor antigens (e.g., MAGE protein family (MAGE-1, MAGE-2, MAGE-3); MART-1 (peptide 27-35); and gp100); and the Colon carcinoma antigens (e.g., peptides of the mutated APC gene product). Particularly preferred Melanoma tumor antigen sequences are those reported by Slingluff et al., in Curr. Opin. in Immunol., 1994, 6:733-740.

The invention in other aspects includes pharmaceutical compositions that are target antigen-specific immune cell response enhancing. Such compositions may be combined, optionally, with a pharmaceutically-acceptable carrier. The term “pharmaceutically-acceptable carrier” as used herein means one or more compatible solid or liquid filler, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

When administered, the pharmaceutical preparations of the invention are applied in pharmaceutically-acceptable amounts and in pharmaceutically-acceptable compositions. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The invention will be more fully understood by reference to the following examples. These examples, however, are merely intended to illustrate the embodiments of the invention and are not to be construed to limit the scope of the invention.

### EXAMPLES

### Example 1

Rapid Early Onset Lymphocyte Cell Death in Mice Resistant but not Susceptible to Leishmania Major Infection

Leishmania major (Lm) is an intracellular protozoan parasite which causes spontaneously resolving cutaneous leishmaniasis in humans. Lm infects macrophages in its vertebrate hosts, and multiplies in the phagolysosomal compartment that contains MHC class II molecules. Consequently, Lm peptides are presented primarly by MHC class II molecules, and evoke a predominantly CD4⁺ T cell response. Experimental cutaneous leishmaniasis in mice has been used extensively as a model for the study of in vivo CD4⁺ T cell differentiation into polarized Th1 and Th2 populations. Th1 and Th2 populations, are characterized by specific, well defined cytokine secretion profiles, as described elsewhere herein, each of which downregulates the other, producing a polarized cytokine bias. In mice, the cytokine bias of the response to Lm infection is generically determined and regulates diseases resistance or susceptibility. Resistant strains, including CBA, C57BL/6, C57BL/10, Sv129 and C3H, produce Th1 cytokines, in particular gamma-interferon (IFNγ), IL-2, and tumor necrosis factor (TNF), in response to Lm infection. Infection in resistant mice is limited to a local cutaneous lesion, which remains relatively small and resolves completely without intervention. The ability of resistant mice to heal the cutanous lesion has been attributed to IFNγ-induced nitric oxide (NO) production by infected macrophages. In contrast, mice from susceptible strains such as BALB/c display Th2 responses characterized by persistent hyper-production of IL-4. The cutaneous lesions in susceptible animals become progressively larger, and eventually the infection spreads systemically and the animal dies. Susceptibility to Lm has been correlated with an IL-4-mediated downregulation of the protective Th1 response, resulting in persistence of the parasite. CD4⁺ T cells, CD8⁺ T cells and B cells all increase in number during infection. Previous reports have shown the interconversion of susceptible and resistant phenotypes by treatment with cytokines and anti-cytokine antibodies, supporting the correlation between cytokine profiles and disease resistance or susceptibility. However, experiments in bone marrow-chimeric mice demonstrate resistance in some animals that secrete Th2 cytokines and generic disruption of the IL-4 gene failed to confer resistance to susceptible BALB/c mice.

Recently, the cell death molecule Fas (CD95) has been implicated in resistance to Lm. Normally resistant mice bearing mutations of Fas or Fas Ligand (FasL) are susceptible to Lm infection despite production of a strong Th1 cytokine response. The mechanism of Fas-mediated disease resistance has not yet been elucidated, and the rile of cell death in disease resistance has never been examined directly. Here, we investigated lymphocyte cell death in resistant and susceptible strains during Lm infection. We show that a massive lymphoproliferative response occurs in the draining lymph node of all infected mice during the first two weeks after infection, but lymphocyte death is observed only in resistant animals during this phase. Subsequently lymphocytes continue to accumulate in susceptible mice by lymphadenopathy of the local node resolves in resistant animals. Furthermore, we demonstrate that Fas is upregulated early during infection in resistant mice, but only later, and to a lesser extent, in susceptible mice. Our findings demonstrate that a delay in the onset of lymphocyte apoptosis and Fas induction correlates with later persistence of lymphadenopathy and susceptibility to disease.

Materials and Methods

Mice and Lm Infection

CBA/J (CBA) and BALB/c mice were obtained from the National Cancer Institute, and bred and maintained in our colony in accordance with national, state and institutional guidelines. Six- to eight week old female mice infected with Lm by injection of 10⁶ (low dose) or 5×10⁶((high dose) stationary phase promastigotes into the hind footpad. These Lm cultures contain between 10 and 20% metacyclic parasites. Lm was a clone of LV39 (Neal or P strain) and was maintained as previously described. (Titus, et al., J. Immunol., 1984, 133:1594-1600). Between three and five mice per strain per time point were infected for each of three experiments.

Quantitation of Cellularity and Cell Death

The popliteal lymph node, which drains the footpad, and the spleen were removed from each mouse 1, 2, 4, or 6 weeks after infection with Lm, or from uninfected control mice (week 0). Lymphoid organs were made into individual single cell suspension, or pooled by group in some experiments. Cells were counted to determine organ cellularity. Cell death was quantitated by enumerating cells stained with Trypan Blue, and verified by ethidium bromide arid Annexin V-FITC Labeling (Apoptest™-FITC, NeXins Research BV, the Netherlands) and analysis by flow cytometry (Coulter Elite Epics, Coulter Corp., Hialeah, Fla.).

In Situ DNA Fragmentation (TUNEL) Assay

Popliteal (draining) lymph nodes were harvested from BALB and CBA mice one week after infectrion with 10⁶ Lm promastigotes. The nodes were fixed in formalin for 24 hours then imbedded in paraffin and cut in 5 gm serial sections. One set of sections was routinely stained with H&E. The other sections were processed for TUNEL staining to identify cells with fragmented DNA (apoptotic cells), as described. (Ahaja, et al., Develop. Genet., 1997, 21:258-267; Zakeri, et al., Biochem. Cell Biol., 1994, 72:603-613. In brief, tissue sections were postfixed with EtOH:HOAc (2:1) and endogenous peroxidase were inactivated with 0.3% H₂O₂. After two washes in 1×PBS, sections were treated with equilibration buffer containing terminal deoxynucleotide transferase (TdT) for 5 minutes, then incubated with reaction mixture containing digoxigenis-11-dUTP (ApopTag Kit, Intergen, Purchase, N.Y.) to label free ends of DNA. Labeling was detected by treatment with anti-digoxigenin-Ab-peroxidase followed by diaminobenzidine. Sections were counterstained with methyl green. Slides were mounted in Permount (Fisher-Scientifc, Pittsburg, Pa.), and photographed at 40× and 100×magnification. Negative control slides were prepared in parallel but the TdT enzyme was omitted.

Determination of Fas Expression by Flow Cytometry

Single cell suspensions prepared from draining popliteal lymph nodes of control and infected mice were labelled with anti-Fas-PE (clone Jo-2, PharMingen, San Diego, Calif.) and fluorescence was quantitated by flow cytometry (Coulter Elite Epics). The mean fluorescence intensity (MFI) was determined for each sample by analysis with CellQuest software (Beacon Dickinson, San Jose, Calif.). Fas induction during infection was calculated by subtracting the mean fluorescence intensity (MFI) or Fas on lymphocytes from uninfectedmice, from the MFI of lymphocytes obtained at each point after infection (ΔMFI=MFI_{week n}−MFI_{uninfected}).

Results

Massive Lymphadenopathy Occurs in the Draining Lymph Node of Lm Infected Mice

All mice infected in the footpad with Lm promastigotes developed cutaneous lesions and a massive increase in cellularity in the popliteal lymph node, which drains the footpad. Footpad lesions resolved in resistant mice but became progressively more extensive in susceptible mice. In parallel, lymphadenopathy of the draining node resolved in resistant animals, but persisted in susceptible mice. We quantitated lymph node cellularity by counting chc cells in the popliteal lymph node at 1, 2, 4 and 6 weeks after inoculation of the footpad with Lm. The response involved CD4⁺ and CD8⁺ T cells and B cells, and their relative proportions in the lymph node changed minimally during the course of infection. This is consistent with other reports in the literature. During thc first two weeks after infection, the kinetics of lymphoaccumulation in the draining node were essentially identical in susceptible and resistant mice. Between weeks 2 and 4 after infection, however, the care on in susceptible mice was 3 to 5-fold greater than in resistance mice (20×10⁶ cells/week compared with 6.9×10⁶ cells/week during low dose infections, and 38×10⁶ cells/week compared with 7.5×10⁶ cells/week during high dose infections). In the final two weeks of infection, lymphoaccumulation continued at only a slightly reduced care in susceptible mice, and mice began to succumb after six weeks of infection. In contrast, lymph node cellularity decreased toward normal in resistant: mice. During this phase, the cutaneous lesion in the footpad resolved in resistance mice but became progressively larger in susceptible animals. Thus, lymphocyte accumulation occurs at the same and to the same extent in the draining lymph nodes of susceptible: and resistant mice for the first two weeks after infection. Thereafter, a dramatic difference became apparent, reflecting an increase rate of lymphocyte accumulation in susceptible mice and a slower or decreased rate in resistant mice. Lm infection via the footpad is normally a cutaneous, localized infection with minimal systemic involvement, reflected by the lack of significant changes in absolute numbers of cells observed in the spleens of infected mice. The infection can spread systemically, however, as demonstrated by the sudden increase in spleen cell number seen in susceptible mice six weeks after infection with a high dose of Lm promastigotes.

Delayed Onset of Lymphocyte Cell Death Correlates with Susceptibility to Lm

Lymphoaccumulation in the draining lymph node corresponds to the sum of cell recruitment into the node, proliferation in situ, and cell death. We determined cell death by staining freshly prepared lymph node cell suspensions with Trypan Blue dye to determine viability of immediately ex vivo cells, and confirmed these results by labeling aliquots of the cells with Annexin V-FITC as a market of apoptotic death. This provided a quantitation of cell death in the lymph node at a given time, and, consequently, the net rate of cell death during each week of infection could be determined. During the first two weeks of infection, a dramatic difference in the rate of cell death was observed between resistant and susceptible mice. In resistant mice, cell death in the draining lymph nodes increased rapidly during the first two weeks, while very little cell death was observed in the nodes of susceptible mice. Both the percent and the absolute numbers of dead cells in the draining lymph node were determined. Up to 25% of cells in the draining lymph node of resistant mice were dead at one week post-infection, while less than 1% of cells were dead in susceptible animals. By two weeks after infection, when the total lymph node cellularity was still essentially identical in the two strains, 5- to 6-fold more cells were dead in the lymph nodes of resistant than susceptible mice. The rate of cell death was higher in resistant mice but the rates of lymphoaccumlation was identical in the two strains, indicating that the rate of proliferation and/or recruitment was also higher in resistant mice. Thus, during the first two weeks after Lm infection, resistant mice demonstrated accelerated cell death accompanied by accelerated proliferation in the draining lymph node. In contrast, susceptible mice displayed a delayed onset of cell death in the draining node. After the first two weeks, cell death leveled off in resistant mice but accelerated in susceptible animals, demonstrating opposite patterns of cell death in the two strains.

In the spleens, there was no significant: change in the percentage of dead cells throughout the course of infection, and an increased number of dead cells was observed only in the spleens of susceptible mice 6 weeks after infection, corresponding to an increased absolute cell number at this time point. Thus, the differential kinetics of cell death between resistant and susceptible strains was specific to the draining lymph node during Lm, infection.

In Situ Apoptosis in the Draining Lymph Node is Extensive in Resistant Mice and Minimal in Susceptible Mice Early in Lm Infection

We examined apoptotic cell death in situ in the draining lymph node of Lm infected mice by the TUNEL technique. TUNEL staining involves the incorporation of labeled nucleotides onto the ends of broken DNA strands, revealing cells with multiple strand breaks that likely underwent apoptotic death. TUNEL staining confirmed that many more cells were apoptotic in the draining lymph node of resistant CBA than of susceptible BALB mice one week after infection with Lm. Control staining was uniformly negative. Thus, histological analysis confirmed that cell death was extensive in the draining lymph nodes of resistant mice, and minimal in those of susceptible mice, early after infection. Furthermore, the lymph node cells in resistant mice were dying by apoptosis.

Fas is Induced More Rapidly and to a Greater Extent on Draining Lymph Node Cells from Resistant Mice

We compared the level of Fas expression and the extent of Fas upregulation on lymphocytes from resistant versus susceptible mice. Although the baseline Fas expression level was higher on cells from susceptible BALB/c mice than on their resistant CBA counterparts, cell surface Fas was upregulated much earlier during infection on cells from resistant animals. One week after infection, Fas expression was only slightly increased in cells from susceptible mice, while Fas expression peaked on cells from resistant mice between one and two weeks after infection. Interestingly, peak Fas expression and maximal rate of cell death coincided during the first two weeks of infection in resistant mice. In contrast, maximal Fas expression and numbers of dead cells were both observed at four to six weeks after infection in susceptible mice. Lymph node cells harvested from the first two weeks of infection from susceptible and resistant strains died in response to anti-Fas antibody in vitro, presumably due to activation induced cell death. Thus, Fas induction was closely correlated with apoptotic cell death in vivo in the draining node in both strains, but the kinetics of Fas expression and cell death were strikingly different between resistant and susceptible mice.

Discussion

Our results demonstrate that lymphocyte apoptosis occurs early in the course of Lm infection in genetically resistant mice, and that cell death in these animals is coincident with rapid cell surface Fas induction. In contrast, cell death and Fas induction are delayed in susceptible mice, which develop massive accumulation of lymphocytes in the draining lymph node and progressive, inexorable lesion growth. These findings suggest that lymphocyte cell death is required for disease resolution. In support of this notion, numerous experiments have shown that a block in cell death pathways, whether by treatment with antibodies, by endogenous mutations, or by targeted gene disruption, results in a susceptible phenotypes. Blockade of CTLA-4 (a molecule implicated in cell death in T cells), resulted in conversion of resistant mice to a susceptible phenotype. (Murphy, et al., J. Immunol., 1997, 159:4460-4469; Corry, et al., J. Immunol., 1994, 153-4142-4148). TNF receptor p55⁻/⁻ mice failed to heal leishmanial lesions, which remained infiltrated with lymphocytes although they were cleared of parasites (Scott, et al., Immunol. Res., 1998, 17:229-238; Vieria, et al., J. Immunol., 1996, 157:827-835). TNF has been implicated in peripheral lymphocyte apoptosis, and cell death appears to be prevented in the lymphocytes infiltrating the lesions, resulting in chronic inflammation and a non-healing phenotype. (Scott, et al., supra; Vieria, et al., supra). In addition, as previously discussed, mice bearing a mutation in Fas or FasL were susceptible to Lm despite Th1 cytokine production. (Huang, et al., J. Immunol., 1998, 160:4143-4147; Conceicao-Silva, et al., Eur. J. Immunol., 1998, 28:237-245). Although Fas-mediated macrophage apoptosis has been suggested as a mechanism for Fas-dependent resistance to Lm (Huang, et al., supra; Conceicao-Silva, et al supra; Moore, et al., J. Immunol., 1994, 152:2930-2937), our data suggests that defective lymphocyte apoptosis leads to persistent inflammation and defective lesion resolution. Defective lymphocyte apoptosis could therefore lead to the release of macrophage-protective cytokines. Collectively, these results teach that macrophage death is required for parasite clearance but early lymphocyte death may be crucial for downregulation of inflammatory cytokine cascades and ultimately, for lesion resolution.

Cytokine bias is undoubtedly critical in resistance versus susceptibility to Lm. However, cytokine bias may regulate disease outcome through the effect of cytokines on lymphocyte cell death. In culture, Th1 effectors die earlier than Th2 effectors (Zhang, et al., J. Exp. Med., 1998, 185:1837-1849). Thus, a Th1 cytokine environment contributes to rapid, early death, for example TNF-induced death. (Zhou, et al., J. Immunol. 1996, 156:2661-1665; Sytewu, et al., Immunity, 1996, 5:17-30; Zhang, et al., J. Exp. Med., 1998, 185:1837-1849). Conversely, Th2 cytokines, including IL-4 and transforming growth factor beta (TGFβ), have anti-apoptotic effects on lymphocytes. (Zhang, et al., J. Exp. Med., 1995, 185:699-709; Swain, et al., Immunol Rev., 1996, 150:143-167; Vella, et al., Proc. Natl. Acad. Sci. USA, 1998, 95:5810-5815; Vella, et al., J. Exp. Med., 1997, 186:325-330; Foote, et al., J. Exp. Med., 1998, 187:847-853; Foote, et al., J. Immunol., 1996, 157:2749-2753; Zamorano, et al., J. Immunol., 1996, 157:4926-4934). It is noteworthy that BALB/c mice express elevated levels of TGFβ (Bogdan, et al., Im. J. Parasitol., 1998, 28:121-134), which may account for prevention of cell death and disease susceptibility in BALB/c IL-4 knockout mice. Chronic cytokine production, be it Th1 or Th2, inhibits healing. (Arkins, et al., Mol. Endocrinol., 1995, 9:350-360; Buck, et al., Am. J. Pathol., 1996, 149:195-204; Cooney, et al., J. Trauma, 1997, 42:415-420; Shi, et al., Proc. Natl. Acad. Sci USA, 1997, 94:10663-10668; Varga, et al., Ann. Int. Med., 1995, 122:60-62). Although sequential regulated cytokine secretion appears to play an important role in wound healing in general, (Hubner, et al., Cytokine, 1996, 8:548-556), chronic or hyperproduction of either Th1 or Th2 cytokines is damaging (Arkins, et al., supra; Buck, et al., supra; Cooney, et al., supra; Shi, et al., supra; Varga, et al., supra). Th1 cytokines, often directly cytotoxic, have been implicated in tissue damage in chronic inflammatory autoimmune diseases and graft-versus-host disease, Nestel, et al., J. Exp. Med., 1992, 175:405-413; Probert, et al., J. Neuroimmunol., 1997, 72:137-141; Isomaki, et al., Ann. Med., 1997, 29:499-507), while Th2 cytokines promote fibrosis and scleroderma-like lesions. Shi, et al., supra; Varga, et al., supra). Normally, production of Th1 cytokines during leishmaniasis may result in resistance because Th1 cytokines promote lymphocyte cell death and thus, limit their own production. (Zhou, et al., J. Immunol. 1996, 156:2661-1665; Sytewu, et al., Immunity, 1996, 5:17-30; Zhang, et al., J. Exp. Med., 1998, 185:1837-1849; Swain, et al., Immunol. Rev., 1996, 150:143-167). When exogenous manipulation (antibody treatment, gene disruption) prevents cell death, the correlation between a Th1 response and disease resistance breaks down, and a pattern of susceptibility in the presence of persistent Th1 response emerges. Thus, an environment permissive for cell death seems to be a unifying factor in successful disease resistance.

### Example 2

Cell Death as a Regulator of Cytokine Bias

Evidence for a regulatory role being played by death of peripheral T cells has come in large part from studies of mouse strains that have lymphoproliferative defects. These mice accumulate large numbers of lymphocytes and manifest dysregulated immune responses. The study of these mice, termed lpr, for lymphoproliferative, and gld, for generalized lymphoproliferative disorder, led to the discovery of the molecules responsible for their phenotype, Fas (CD95) and FasL (CD95L), respectively. Study of how these molecules shape an immune response has been explosive. It is now clear that Fas and FasL are actively involved in apoptotic death of peripheral lymphoid, and potentially other, cells.

Lymphocyte death can involve other receptor ligand pairs, including the CD4 molecule on the CD4⁺ T cell and its ligand the major histocompatibility complex (MHC) class II molecule. The CD4³⁰ molecule on the T cell binds the non-polymorphic region of MHC class II molecules on the MHC class II bearing cell. This interaction is thought to augment T cell receptor (TCR)-mediated recognition. The interplay between costimulation and cell death has yet to be elucidated.

Costimulatory interactions that result in lymphocyte activation are paracrine, resulting from the interaction between cells. Likewise, cell death can result from paracrine interactions, e.g. cells bearing FasL, such as a CD8⁺ T cell or a natural killer (NK) or γδ T cell, kill Fas bearing targets. Therefore, costimulatory interactions and death-inducing receptor/ligand interactions must be coordinately regulated. Lymphocyte death can also be autocrine, as a single cell bearing both Fas and FasL has been demonstrated to die alone. These observations leave open the possibility that during the initiation and expansion phases of an immune response, paracrine “immune-directed” death results in a selective advantage for those cells receiving the appropriate costimulation, and thus leads to cytokine bias by selective cell survival. Once the expanded population of cells is no longer receiving an antigen-driven survival advantage and paracrine costimulation, autocrine death may provide a mechanism for the removal of antigen specific cells which are no longer needed.

Normal Mouse T Cells Express IE

Previous work with human T cells indicates that activation of the T cells by antigens or engagement of CD4 results in expression of HLA DR on the T cell surface. Expression of MHC class II on mouse T cells is controversial. Reports indicate both positive and negative results. The studies to date have not distinguished between failure to express IA versus IE. To address the possibility that normal mouse T cells express IE we isolated lymph nodes or spleens from strains of animals which express IE (Balb/c, CBA, and AKR mice). Spleens or nodes were taken from 4 week old mice, minced to single cell suspension, and red blood cells were removed via Gey's treatment. Splenocytes were then passed over Cellect Columns (Cytovax, Edmonton, Canada) to purify CD4⁺ T cells. CD4⁺ T cells were collected, found to be 98.5% pure, and contaminants were identified flow cytometrically as NK and γδ T cells.

The CD4 T cells were treated with antibody to CD4 (GK1.5) at 10 mg/ml/10 7 cells. washed and treated with rabbit anti-rat antibody for 45 minutes at 37° C., followed by washing. The cells were cultured overnight and stained with FITC conjugated anti-IE antibody (14-4.4 S), above, or 14-4.4S and counterstained with anti-TCR.

For the PCR experiment below, purified CD4⁺ T cells, 5×10⁶/ml, were incubated for 8 hrs. with biotinylated antibodies for CD4 (GK1.5), CD28, CD3 (145.2C11) alone, CD4 and CD28, CD3 and CD28, or no treatment. Experimental setup included wells of purified T cells and percoll-isolated B cells added to control for potential MHC Class II⁺ contaminants. B cells, 5×10⁵ cells, which is 5% of purified T cells (far greater than the 1.5% contaminants seen following Cellect Column purification), were added to T cell wells. Cells were then washed, collected and total RNA was isolated using an RNA isolation kit, RNEasy (Qiagen, Chatsworth Calif.). Single strand DNA was generated from 2 mg of RNA using SuperScript II reverse transcriptase (GIBCo/BRL Gaithersburg, Md.). PCR was done using MHC Class II, (I-E, exon 3 primers). PCR protocol was: 1 min at 94° C., 1 min at 60° C., and 2 min. at 72° C. for 35 cycles. Following PCR, samples were loaded onto 1% agarose gels, stained with ethidium bromide and visualized with UV light.

MHC Class II Mediated Cell Death

Because one possibility is that class II molecules deliver death signals to the Th2 cells either directly or indirectly, experimental data obtained (see below) demonstrates that MHC class II engagement on resting, but not on primed or activated, B cells results in the apoptotic death of the B cells. Also shown, is that the treatment of any of the populations of B cells with isoproterenol, a β-adrenergic agonist which elevates cAMP, induces apoptosis in all three populations and was therefore used as a standard for normalizing apoptosis induced by the other stimuli.

Splenic B cells from AKR mice were separated on a Percoll density gradient and two fractions were collected: small, high-density, resting B cells were obtained from the 1.079<ρ<1.085 g/ml interface and large in vivo activated B cells were collected at the BSS<ρ<1.066 g/ml interface. In vitro primed B cells were obtained by treating resting B cells with 16U recombinant IL-4 and by incubating them overnight on anti-Ig-coated plates. Each population was treated with 10 μg/ml of anti-class 11 mAb (10-2.16), with 10 μg/ml of anti-Ig plus 16U rIL-4, or with 10 μM isoproterenol for 10 min at 37° C. and then was incubated overnight. Apoptotic death was scored by quantitating the formation of nucleosome-sized DNA fragments by densitometry.

Analysis of the Molecular Mechanism of Class II-Mediated Apoptosis

Generation of cAMP in B Cells

We have analyzed the molecular mechanisms of class II-mediated apoptosis. Previous kinetic analysis had revealed that the short term levels of intracellular cAMP continued to rise until 10 min post class II crosslinking, so a 10 min time point was used in subsequent experiments. As shown above and elsewhere, high-density resting, but not anti-Ig plus IL-4 primed or in vivo-activated, B cells respond to anti-class II treatment by elevating cAMP.

The primed/activated cells do not undergo class II-mediated apoptosis even though they die when treated with isoproterenol indicating that the death inducing machinery downstream of the generation of cAMP is functional. We have subsequently carried out detailed analysis of both kinetics and dose-response of cAMP elevation in resting B cells after treatment with anti-class II mAbs. These results demonstrate that an initial short burst of cAMP peaking around 8-10 min post stimulation is sufficient to initiate the death cascade in these B cells. We also determined cAMP levels at 6 h and 24 h post stimulation. At these two time points the cAMP levels were equal to those from untreated cells; however, the low viability in the cultures at these later time points made the interpretation of these data problematic. Although previous studies had indicated that a 10 μg/ml dose of anti-class II mAb was optimal for elevating cAMP in resting B cells from AKR mice, we observed that this dose had little effect on resting B cells from C57BL/6 mice. We therefore carried out a full dose-response profile with B cells from the latter strain and discovered that maximal cAMP is obtained at a 100-fold lower dose of mAb than is optimal for AKR B cells. In addition, the dose-response of the C57BL/6 B cells to anti-class II mAb shows a curious oscillatory pattern and further analysis will be required to elucidate both the strain variation in dose-response and the mechanistic basis for these oscillation of response.

cAMP response in B cells: Splenic B cells from AKR mice were prepared and treated as described earlier. At the end of the 10 min, 37° C. treatment period, the cells were lysed in ice cold acidic ethanol. cAMP levels were determined by RIA using a commercial kit (Amersham). Results are expressed as fold increases over levels in untreated controls to allow multiple experiments to be averaged. Means of three independent experiments and standard errors of the mean are shown except for the primed B cells for which the means of two independent experiments are shown. Resting B cells, in vivo activated B cells, and in vitro primed B cells were analyzed. Resting B cells were treated with 10 μg/ml of anti-class II mAb or with 10 μM isoproterenol, and then were lysed in acidic ethanol. cAMP levels were determined as before. Resting B cells were prepared and were treated with various doses of anti-class II mAb for 10 min at 37° C. cAMP was determined as described above.

Role of CD95 (Fas) in Class II-Mediated Cell Death

The following data was generated to elucidate the relationship between MHC class II mediated cell death and CD95 (Fas). Studies on the mechanisms of cell death have revealed that any cells, including B cells, do not always die by apoptosis (or necrosis) but can die by a process known as “oncosis,” under certain circumstances. Experimentally, oncosis is distinguished from apoptosis by the following rapid (<1 h) processes: a decrease in forward scatter, incorporation of ethidium bromide or propidium iodide (without treating the cells with saponin), and by the lack of DNA fragmentation (measured by DNA gels, TUNEL or saponin/PI).

We have recently developed tumor models for both the dividing (mitotic) and growth arrested (post mitotic) or growth arrested primary cells. These cells have been used to study the biochemistry of class II-associated proteins. The mitotic cell model is the BALB/c-derived B cell lymphoma L1210 and the growth-arrested, post-mitotic cell model is L1210/DDP, a multi-drug resistant subline of L1210. Both of these cells were characterized for the expression of class II, CD95, B7-1 and B7-2 and for their ability to die when treated with anti-class II mAb or the drug methotrexate. The L1210 cells are positive for CD95 and B7-1 and express very low levels of class II and B7-2. Intracellular Bc1-2 was not detected above background staining. Conversely, the L1210/DDP cells express high levels of the B7 molecules and Bc1-2, but are essentially negative for CD95.

L1210 and L1210/DDP cells were treated overnight with 5×10⁻⁷ M methotrexate. Flow cytometric analysis revealed two populations based on forward side scatter. The forward scatter high populations did not take ethidium bromide, and were therefore considered viable. The forward scatter low populations took up ethidium bromide differentially. The L1210 cells took up a moderate amount of ethidium bromide whereas the L1210/DDP cells took up large amounts. Analysis of DNA fragments revealed that L1210 produced a ladder of nucleosome sized fragments indicative of apoptosis, whereas L1210/DDP cells did not. This latter phenotype—loss in forward scatter and membrane permeability with no “DNA laddering”—is the hall mark of oncosis.

L1210 and L1210/DDP cells were treated with a dose of methotrexate that produced moderate amounts of cell death. (Even though L1210/DDP is “methotrexate resistant”, the drug does induce cell death in the cultures; the surviving cells which grow out of the culture are responsible for the lines “drug resistant” appellation). As described earlier, L1210 cells die by apoptosis, as indicated by the drop in forward scatter, the moderate uptake of ethidium bromide and the generation of nucleosome sized fragments of DNA. L1210/DDP cells die by oncosis, as indicated by the drop in forward scatter (with a more pronounced loss of side scatter), very high uptake of ethidium bromide and no DNA fragmentation.

Resting, primed and activated splenic B cells from AKR mice were prepared as described above. Cells were stained with FITC anti-CD95 or with isotype control. As described below, resting, in vitro primed and in vivo activated cells, all express modest amounts of CD95, with the activated cells expressing slightly less than the other two populations. This is consistent with the use of the L1210/DDP lymphoma as a model of this cell type. In light of the CD95-dependent apoptosis that we have observed, we needed to establish whether a functional link between class II and CD95 existed. First, we demonstrated the ability of class II to regulate CD95 expression. For these studies, we isolated B cells as described and treated the cells with antibodies to class II molecules. The engagement of class II molecules with soluble mAb was insufficient to induce CD95 expression. However, when I-A molecules on resting B cells were oligomerized using a biotinylated antibody followed by streptavidin, were incubated overnight, and were stained for CD95 using fluorescein conjugated anti-CD95 (Jo2, Pharmingen), they were induced to express high levels of CD95 (not shown). These data are consistent with the notion that dimeric engagement of class II molecules is insufficient to alter CD95 expression, but that extensive oligomerization of class II molecules can induce CD95 expression.

Next we tested the hypothesis that the mechanism of class II mediated apoptotic death involves the receptor ligand pair CD95/CD95L. Toward this end, we used mouse strains that have the lpr mutation or the gld mutation, which have defects in CD95 and CD95L, respectively. Total splenic B cells were isolated from the normal strains C3H and AKR, and from the C3H.lpr, and MRL.gld strains (All of these strains are H-2k). Whereas class II crosslinking on B cells from C3H and AKR animals caused a significant increase in apoptotic cells (<2N DNA, or “hypodiploid”), similar treatment caused only a marginal increase in apoptotic cells from the C3H.lpr and MRL.gld animals. The observation that the CD95 and CD95L mutant mice showed any apoptosis above background suggests that, while class II-induced apoptosis in these cells is primarily CD95 dependent, there is a component of class II-mediated apoptosis that is CD95 independent.

Total splenic B cells were isolated from C3H, AKR, C3H.lpr, and MRL.gld mice. The cells were cultured overnight, harvested, permeabilized in saponin, stained with propidium iodide (PI) which intercalates into DNA, and analyzed by flow cytometry. Because the majority of cells in these preparations are either resting or “growth arrested,” the predominant peak in the untreated samples is the G₀/G₁ peak (2N DNA). However, after 15 h in culture, a significant percentage of B cells fragmented their DNA, as indicated by the broader peak to the left of the G₀/G₁ peak (“hypodiploid”, <2N DNA). Crosslinking class II on B cells from the wild type animals (C3H and AKR) caused an increase in apoptosis as indicated by the increase in the <2N DNA peak. Unlike the normal B cells, there is only a modest increase in the <2N DNA after crosslinking MHC class II on B cells from lpr or gld mice. The panels showing emetine treated cells demonstrate that B cells from lpr animals are equally capable of fragmenting DNA as B cells from the normal animals. The PMA plus ionomycin control demonstrates that in appropriately stimulated cells one obtains both a reduction in cells with fragmented DNA and an increased proportion of cycling cells (DNA>2N).

Based on these results we addressed the possibility that class II engagement regulated the ability of CD95 to function as a death-inducing molecule in B cells. We isolated resting or in vivo activated (growth arrested) cells, treated them with anti-class II antibodies, and cultured the cells overnight on plates that had been coated with anti-CD95 or not. The results, summarized in the Table 1 below, suggest that in resting B cells class II and CD95 signals synergize to cause increased apoptotic death. (Even though this table uses drop in forward scatter—a technique that cannot distinguish apoptosis from oncosis—to measure cell death, a parallel experiment used TUNEL analysis to show that the death in resting B cells was, indeed, apoptotic). Anti-CD95 treatment has no effect over background on the activated cells; however, anti-class II treatment induces significant death, presumably by oncosis (drop in forward scatter shown here combined with the lack of DNA fragmentation shown). Recent work, and our own data suggest that mechanism by which class II augments CD95-mediated death in resting B cells is that class II engagement not only up-regulates expression of CD95 but also induces the expression of CD95L and the cells die as a consequence.

TABLE 1
Class II induces CD95-dependent death.

| | % Dead Cells¹ | | | |
|---|---|---|---|---|
| | | Resting + | | Activated + |
| Treatment | Resting | Anti-CD95 | Activated | Anti-CD95 |
| Control | 27 | 36 | 58 | 61 |
| Anti-Class II | 43 | 57 | 84 | 89 |

¹Determined by loss in forward scatter, a technique that does not differentiate between apoptosis and oncosis in primary cells.

In a preliminary experiment, L1210 and L1210/DDP were cultured overnight on plates coated with MK-D6 (anti-I-A^{d}, the cells are BALB/c-derived). Cell death was scored by loss of forward scatter. Cell viability for the L1210 was not significantly affected by the anti-class II (87% viable for the control, 88% for the treated culture) supporting their use as a model for anti-Ig plus IL-4 primed cells. Cell viability for the L1210/DDPs dropped from 75% in the untreated cultures to 48% for those treated with anti-class II mAb.

### Example 3

Cell Death as a Regulator of Cytokine Bias (Continued)

Physiological cell death occurs through the processes of apoptosis and necrosis. The boundaries between these processes, once thought to be distinct, have blurred with the explosion of information on the role of cell death in development, tissue modeling, regenerative processes, and in the immune system. However, it is widely accepted that necrotic cell death typically results in the osmotic rupture of a cell, followed by an inflammatory response, while apoptotic death involves cell shrinkage, fragmentation of the cell, and phagocytosis of the fragments without inflammation. Most cells die in a form of suicide characteristically apoptotic and tightly regulated by complex signals. Apoptotic cell death is particularly important in the reticulo-endothelial system where the balance between mitosis and cell death may determine the effectiveness and the nature of an immune response. Failure results in autoimmune disease or in a lack of immune surveillance.

Selective Death of Antigen Presenting Cells as a Factor in Immune Deviation

Death at various stages of the immune response has previously been documented but its role in immune deviation has not yet been determined. Selective death of antigen presenting cells (APC) may influence immune deviation since infectious microorganisms can either induce or inhibit apoptosis of macrophages or dendritic cells. Salmonella typhimurium induces apoptosis of infected macrophages, and infection with Legionella pneumophila, the causative agent of Legionnaires disease and Pontiac fever, induces apoptosis of monocyte cell line HL60. Apoptosis of macrophages in Shigella and Bordatella pertussis infections is required for inflammatory pathology, suggesting a link between APC cell death and the type of immune response which is initiated. In contrast, Candida albicans infection can inhibit TNFα mediated apoptosis of the monocytic cell line U937 or human peripheral blood monocytes. Taken together these results suggest that apoptosis of the monocyte or macrophage may be important for initiation of inflammatory processes, infection, bacterial survival, escape from a particular type of immune response, and potentially in determining the type of immune response that occurs. Evidence for the latter is provided by the finding that Th1 CD4⁺ T cells induce apoptotic death of susceptible antigen presenting cells. The question remains as to how the death of the APC or a subset of APC influence the initial steps in T cell priming and cytokine bias. One of the ways in which APC death may be regulated is via signaling through MHC-encoded molecules, as we have found that MHC class II engagement can mediate cell death.

CD4⁺ T cells recognize antigens which are associated with MHC class II molecules. Many investigators have established that engagement of MHC class II molecules results in signals being delivered through the class II molecules to the cell on which the molecules are expressed. Furthermore, MHC class II engagement delivers distinctly different signals to the cell depending on the activation state of the cell and on the structural conformation of the MHC class II molecules, which couples engagement to different signaling cascades. We and others have observed that CD40 primes B cells for Fas-induced apoptosis unless the B cell is rescued from Fas-induced death by antigen receptor rescue. We have observed that activated γδ T cells express CD40 ligand constitutively. We have confirmed with flow cytometric analysis showing changes in cell surface expression of IE on immediately ex vivo CD4⁺ T cells from the transgenic B1TgE or Balb/c after CD4 engagement.

We established that MHC class II engagement can result in death of B cells (see Example 2 above). Furthermore, MHC class II mediated cell death involves members of the family of death-inducing receptor/ligand pairs, Fas (CD95) and FasL (CD95L). Data from our laboratory illustrates that this phenomenon can be extended to MHC class II bearing monocytic cells.

The level of expression of MHC class II on APCs is regulated in large part by cytokines and other extracellular mediators. Several studies have now suggested that production of nitric oxide (NO) is the critical factor that determines the level of expression of MHC class II in macrophages in response to products of bacterial pathogens. NO is derived from molecular oxygen and the guanidino nitrogen of L-arginine in a reaction catalyzed by NO synthase (NOS). CD4⁺ Th1 cells secrete interferon-gamma (IFNγ), which is known to induce NO production by macrophages. In murine leishmaniasis, macrophages activated with IFNγ or TNFα produce high levels of NO and are strongly leishmanicidal. This process is inhibited in the presence of IL-4. NO, as well as IFNγ and TNFα, is also known to be induced during the course of murine viral myocarditis induced by Coxsackie virus B3 (CVB3). When macrophages are exposed to bacterial products such as LPS, LPS-induced TNFα leads to NO production and MHC class II expression is inhibited. If TNFα does not stimulate NO production in the macrophage, MHC class II expression is augmented by LPS. Thus, the production of NO may protect APCs from MHC class II mediated death signals. The coordinate regulation of life or death of subsets of APCs thus promotes the priming of T cells with a particular cytokine bias.

Regulatory Interactions Resulting in Death of CD4⁺ T Cell Subsets

Once priming of T cells has occurred, activated CD4⁺ T cells proliferate. The selection process which determines the profile of the cytokine produced is not known. What is clear is that Th1 cells produce IL-2 and IFNγ, cytokines which potentiate cell-mediated, delayed type hypersensitivity (DTH) immunity; Th2 cells characteristically secrete IL-4 and IL-10, cytokines involved in promoting T-dependent B cell responses that result in high level antibody production (humoral immunity). These two classes of cytokines appear to negatively regulate each other, resulting in the amplification of the initial response type with repression of the other. The cytokine bias of the initial response appears to be dependent on the nature of the antigen, the antigen dose, the type and route of administration of antigen, and by poorly defined genetic factors. Several lines of evidence suggest that CD4 engagement and/or signals may be involved in determining the cytokine bias of the immune response, via CD4-regulated cell death of a CD4⁺ cell subset.

Factors controlling the avidity and affinity of the CD4/MHC class II interaction include the level of CD4 expression, and the allele of MHC class II acting as the CD4 ligand. The genetic background, especially the allele of MHC class II, affects the cytokine bias in the T cell response to antigenic stimuli in vivo, in several mouse systems of parasitic and autoimmune disease. We have chosen two models to investigate the possibility that selective cell death shapes the cytokine bias of a response. In the first, we have studied Leishmania major (Lm) (see Example 1). A second model of cytokine bias determining disease phenotype is Coxsackievirus B3 (CVB3)-induced myocarditis. In this model, virus infection of the myocardium stimulates pathogenic cellular immunity which causes most of the tissue injury associated with the disease. The type of T cell response is a deciding factor in pathogenicity as the difference between a highly myocarditic CVB3 variant (designated H3 virus) and an amyocarditic CVB3 variant (designated H310A1) lies in the ability of the former virus to induce a predominantly virus-specific Th1 response while the latter variant induces predominantly Th2 cell activation in male mice. The genetics of the host affect Th subset selection, although the mechanisms involved here are less clear. H3 virus infection of male BALB/c mice triggers Th1 cell responses and results in disease, while infections of C57Bl mice, which result in equivalent levels of virus in the heart, induce Th2 cell responses and little myocarditis. Finally, while male BALB/c mice are susceptible to H3 virus-induced myocarditis, virgin females are not because estrogens present in the female promote, either directly or indirectly, Th2 cell responses and little myocarditis. Importantly, Th cell subset selection depends largely on whether potent γδ T cell responses are induced as these activated γδ T cells appear to regulate Th cell differentiation. There is evidence suggesting that the role of γδ T cells is to promote immune deviation by preferentially inducing death of Th2 cells.

Antigen Clearance, Death of Immunocytes, and Memory

Given the numbers of lymphocytes that are generated during an immune response, it seems only logical that there must be a way of removing antigen specific T cells as antigen is cleared. In fact, experimental evidence now suggests that many CD4⁺ T cells undergo apoptotic death as the response subsides. However, clearly some cells that respond to the pathogen survive and function as memory cells. CD4⁺ T cell memory is widely thought to be the consequence of T cells responding to persistent antigens. The complex regulation of life and death signals which determine the fate of antigen specific CD4⁺ T cells as antigen is cleared has yet to be characterized. However, recent studies suggest that both T cell receptor mediated activation and T cell activation induced death may involve Fas and FasL. During the course of an immune response, antigen activated T cells express the Fas molecule, but are refractory to Fas induced death for several days. This argues that the presence of antigen may protect the responding T cell from Fas/FasL mediated death. Likewise, growth factor withdrawal which could also result from decreased antigen receptor engagement, results in apoptotic cell death of responding T cells.

Our early work aimed at addressing the role of CD4 engagement in the activation of T cells lead to the discovery that separation of signals generated by CD4 engagement and T cell receptor engagement resulted in T cell receptor mediated death of the T cell. This was followed by the experiments which indicated that parallel processes may account for the loss of CD4⁺ T cells in HIV infection when gp120 from the HIV virus has bound CD4 and subsequent antigen receptor engagement occurs. Extending these observations, our recent work has established that CD4 cross-linking increases the sensitivity of a subset of CD4⁺ cells to Fas-induced death signals, as well as rapidly increasing cell surface Fas expression. Taken together these results suggest that CD4 involvement in an antigen specific immune response, once the antigen is no longer around to provide a life promoting signal through TCR-mediated signals, programs the cell to die.

This model predicts that the nature of the CD4 interaction with MHC class II biases the response of the CD4⁺ T cell and the response can mean life or death of either cell. This bias may result from quantitative differences in the amount or nature of CD4 molecules expressed on the cell surface or the number of CD4 molecules bound. Likewise because CD4 binds to MHC class II, the numbers of MHC molecules per APC or the sequence or structure of the polymorphic MHC molecules may directly impact the number or signals delivered through molecules of CD4 and TCR. Clearly one possible genetic influence is the haplotype of the MHC and the preferential use of MHC class IA versus IE in the presentation of antigens. The number and the nature of the MHC class II molecules engaged is influenced by the antigen and the antigen load.

In summary, we have shown that in the same way that cell death shapes development, cell death shapes immune deviation from initial priming of T cells through the effector function of the response during which the T cell produces cytokines, and finally, to the generation of CD4⁺ memory cells.

### Example 4

MHC Class II is Necessary for the Killing of the B Cells

Materials and Methods

Transgenic mice expressing either wild type MHC class II, or hybrid MHC class II-MHC class I molecules (Kjer-Nielsen L, et al., 1992, Transgenic Res, 1:182-187) on a null MHC class II background (Viville S, et al., 1993, Cell, 72:635-648), were obtained from Dr. Jim McCluskey (Department of Microbiology and Immunology, University of Melbourne, Parkville, Victoria, Australia)

Transgenic mice (known as 3A9) expressing a hen egg lysozyme (HEL)-specific T cell receptor (Ho W Y, et al., 1994, J Exp Med, 179:1539-1549), were obtained from Dr. Mark Davis (Department of Microbiology and Immunology, Stanford University School of Medicine, California).

Animals were kept at UVM's animal facility (accredited by the American Association for the Accreditation of Laboratory Animal Care); all procedures were approved by the Institutional Animal Care and Use Committee.

B Cell/T Cell Isolation

Mouse spleens were removed after sacrifice and placed into 4 ml PBS, 0.1% BSA in a petri dish. A single cell suspension was prepared using the frosted ends of two microscope slides. Cells were then transferred into a 15 ml tube. The cell suspension was underlayed with 2 ml Ficoll-paque and then centrifuged for 15 min. at 1,000×g. Lymphocytes at the interface were collected and washed 3× with ice-cold 15 ml PBS, 0.1% BSA. Cells were counted and resuspended to 1.6×10⁷ viable cells per ml concentration, in RPMI 1640 25 mM HEPES, 50 μM 2-ME, 10% FBS, 1× L-glutamine, 1× sodium pyruvate, 1× nonessential amino acids.

For CD4⁺-enriched cells, spleen cells obtained above were treated with a cocktail of cytotoxic antibodies to CD8⁺, heat-stable antigen, and class 11 as described in Croft M, et al., 1992, J Exp Med, 176:1431-7. For mature B cell -enriched cells, spleen cells were T cell-depleted using anti Thy 1.2, anti CD4, anti CD8 , plus complement (also as described in Croft M, et al., 1992, J Exp Med, 176:1431-7), and positively selected using anti-B220.

Antibodies, Staining and Flow Cytometry

The EZ-link NHS-LC biotin (cat no. 21336) from Pierce Chemicals (Rockford, Ill.) was used for biotinylation (conjugation) of antibodies and antigen. Substrates are biotinylated at a concentration of 110 μg biotin/mg substrate. The reaction is carried out at room temperature for a few hours or overnight at 4° C. Unbound components may be separated by overnight dialysis at 4° C. in PBS. anti-IgM-avidin-HEL Ab: Avidin and HEL from Sigma Chemical Co., St. Louis, Mo. (cat nos. A9275 and L6876 respectively). Molar ratios: 1.0:0.5:3.0 for antibody:avidin:antigen. A 5× concentrated stock in media may be prepared and sterilized by filtration through 0.2 μm syringe filter (cellulose acetate). C4H3 is a monoclonal antibody specific for HEL 46-61 determinant bound to I-Ak (Reis e Sousa C, et al., 1999, J Immunol, 162:6552-61). T cell and B cell specific antibodies were obtained from Pharmingen (San Diego, Calif.).

The FACScalibur, flow cytometer was calibrated using Becton-Dickinson calibration beads prior to each four color analysis run. Cells also were analyzed on a FACScan™ with CellQuest software (Becton Dickinson, San Jose, Calif.).

Results:

Two different crosses between the mice carrying the different transgenes were generated. Transgenic mice expressing a hen egg lysozyme (HEL)-specific T cell receptor (3A9) were crossed with: (i) the transgenic mice expressing wild type MIIC class II on a null MHC class II background, and (ii) the transgenic mice expressing hybrid MHC class II-MCH class I molecules on a null MHC class II background.

Mice, 4-8 weeks old, generated from the above-identified crosses were pulsed in vivo with an injection of 100 μg total of antigen (HEL). Six days after injenction, the cells from splenes were harvested, as described above, and analyzed. Mice from the 3A9×wild type MHC class II on a null MHC class II background cross had no MHC class II bearing B cells, and normal T cell numbers. Mice from the 3A9×hybrid MHC class II—MHC class I molecules on a null MHC class II background cross had MHC class II bearing B cells, and a CD4⁺ T cell subset missing. The results show that the “tail” of MHC class II (missing in the hybrid) is necessary for the killing of the B cells. Additionally, the “tail” of MHC class II is not necessary for the proper loading and presentation of antigen.

### Example 5

T Cell Function Assays

T cell function is evaluated by the proliferative potential to specific and non-specific antigens using standard assays. Specifically, the assay assesses the response of T cell receptor (TCR) mediated proliferation using anti-CD3 antibodies (Becton Dickinson) as well as baseline non-specific proliferation using concavalin A (Con-A). Briefly, T cells are washed and resuspended in RPMI with 10% FCS at a concentration of 10⁶ cells/ml. 100 μl (10⁵ cells) are added to each well of a 96 well plate. Cells are stimulated with either Con-A (5 μg/ml) (non-specific response) or monoclonal antibodies to CD3 in the presence of IL-2 (20 units/ml) and irradiated mononuclear cells (MCs) (10⁵ cells/well in 100 ml of RPMI with 10% FCS). Purified goat anti-mouse F(ab)′2 fragments (Kirkegard and Perry Laboratories, Gaithersberg, Md.) are used as a crosslinking agent for the experimental conditions where monoclonal antibodies to CD3 are used. Wells are pretreated with 1.25 μg/ml of goat anti-mouse antibody for 45 minutes at 37° C. and washed three times prior to the addition of monoclonal antibodies to CD3 and CD28. Controls included T cells alone, T cells plus irradiated MCs, and T cells plus mitogenic stimuli without IL-2 or irradiated MC. After 7 days in culture at 37° C., cell proliferation is assessed using either radio-active assays or commercially available non-radioactive, ELISA based assays (e.g. Promega). Cells are co-cultured for 5-7 days to induce proliferation of the T cells (the stimulator cells are also irradiated and thus non-proliferative). Stimulator cells alone serve as controls.

An additional approach to testing T cell function uses flow cytometry based staining for intracellular expression of the cytokines IL-2, IFN-γ, and TNF-α using antibodies specific to the human forms of these factors (Becton Dickinson). These cytokines are produced in the T progeny in the antigen specific in vitro proliferation assays. Further, semiquantitative RT-PCR of mRNA for these factors can also be used.

Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety.

What is claimed is presented below. We claim:

1. A method of autologous adoptive immunotherapy, comprising:
obtaining B cells that have been originally isolated from a subject and subsequently contacted in vitro with a target antigen conjugate to produce target antigen manipulated B cells, and infusing the target antigen manipulated B cells into the subject,
wherein the target antigen conjugate comprises a target antigen that elicits a Th2 response conjugated to an antibody that selectively binds a B cell cell-surface immunoglobulin.

2. The method of claim 1, wherein the target antigen manipulated B cells enhance an antigen-specific immune cell response to the target antigen that elicits a Th2 response in a subject.

3. The method of claim 1, wherein the isolated B cells are mature B cells.

4. The method of claim 1, wherein the B cells are isolated from peripheral blood or an in vitro hematopoietic progenitor cell culture.

5. The method of claim 1, wherein the in vitro contacting of the isolated B cells with a target antigen conjugate, further comprises contacting the isolated B cells with an agent selected from the group consisting of a B cell co-stimulating agent and a Th2 cytokine.

6. The method of claim 5, wherein the B cell co-stimulating agent is selected from the group consisting of TSA-1, CD2, CD5, CD24, CD28, CD40L, CD49a, CD80, CD81 and Cd86.

7. The method of claim 5, wherein the Th2 cytokine is selected from the group consisting of IL-4, IL-5, IL-6, IL-9, IL-10, and IL-13.

8. A method of autologous adoptive immunotherapy, comprising:
obtaining CD4⁺ helper T cells that have been originally isolated from a subject and subsequently contacted in vitro with a target antigen-conjugate to produce target antigen manipulated CD4⁺ helper T cells, and infusing the target antigen manipulated CD4⁺ helper T cells into the subject,
wherein the target antigen conjugate comprises a target antigen that elicits a Th2 response conjugated to an antibody that selectively binds a T cell receptor.

9. The method of claim 8, wherein the target antigen manipulated CD4⁺ helper T cells enhance an antigen-specific immune cell response to the target antigen that elicits a Th2 response in a subject.

10. The method of claim 8, wherein the CD4⁺ helper T cells are isolated from peripheral blood or an in vitro hematopoietic progenitor cell culture.

11. The method of claim 8, wherein the in vitro contacting of isolated CD4⁺ helper T cells with a target antigen-conjugate, further comprises contacting the isolated CD4⁺ helper T cells with a Th2 cytokine.

12. The method of claim 8, wherein the Th2 cytokine is selected from the group consisting of IL-4, IL-5, IL-6, IL-9, IL-10, and lL-13.

13. A method of autologous adoptive immunotherapy, comprising:
obtaining CD4⁺ helper T cells that have been originally isolated from a subject and subsequently contacted in vitro with a target antigen-conjugate to produce target antigen manipulated CD4⁺ helper T cells, and infusing the target antigen manipulated CD4⁺ helper T cells into the subject,
wherein the target antigen conjugate comprises a target antigen that elicits a Th1 response, conjugated to a bi-specific antibody that selectively binds both a T cell receptor and CD4.

14. The method of claim 13, wherein the CD4⁺ helper T cells are isolated from peripheral blood or an in vitro hematopoietic progenitor cell culture.

15. The method of claim 13, wherein the in vitro contacting of the isolated CD4⁺ helper T cells with a target antigen-conjugate, further comprises contacting the isolated CD4⁺ helper T cells with a Th1 cytokine.

16. The method of claim 15, wherein the Th1 cytokine is selected from the group consisting of IL-2, TNF-α, IFN-γ, and lymphotoxin.

17. A target antigen-specific immune cell response enhancing composition, comprising:
a target antigen conjugated to an antibody that selectively binds a B cell cell-surface immunoglobulin, wherein the target antigen elicits a Th2 response.

18. A target antigen-specific immune cell response enhancing composition, comprising:
a target antigen conjugated to an antibody that selectively binds a T cell receptor, wherein the target antigen elicits a Th2 response.

19. A target antigen-specific immune cell response enhancing composition, comprising:
a target antigen conjugated to a bi-specific antibody that selectively binds both a T cell receptor and CD4, wherein the target antigen elicits a Th1 response.

20. A target antigen-specific immune cell response enhancing composition, comprising:
an isolated B cell contacted in vitro with a target antigen conjugated to an antibody that selectively binds a B cell cell-surface immunoglobulin, wherein the target antigen elicits a Th2 response, and
a pharmaceutically acceptable carrier.

21. A target antigen-specific immune cell response enhancing composition, comprising:
an isolated CD4⁺ helper T cell contacted in vitro with a target antigen conjugated to an antibody that selectively binds a T cell receptor, wherein the target antigen elicits a Th2 response, and
a pharmaceutically acceptable carrier.

22. A target antigen-specific immune cell response enhancing composition, comprising:
an isolated CD4⁺ helper T cell contacted in vitro with a target antigen conjugated to a bi-specific antibody that selectively binds both a T cell receptor and CD4, wherein the target antigen elicits a Th1 response, and
a pharmaceutically acceptable carrier.
